(19) United States
(12) Patent Application Publication
(10) Pub. No.: US 2025/0025393 A1
(43) Pub. Date: Jan. 23, 2025

(54) COSMETIC OR DERMATOLOGICAL COMPOSITION COMPRISING A MEROCYANINE AND DIPROPYLENE GLYCOL

(71) Applicant: L'OREAL, Paris (FR)

(72) Inventors: Flavie GILLANT, Chevilly la Rue (FR); Angélina ROUDOT, Chevilly la Rue (FR); Cécile BOSCHET, Chevilly la Rue (FR)

(73) Assignee: L'OREAL, Paris (FR)

(21) Appl. No.: 18/706,020

(22) PCT Filed: Dec. 12, 2022
(86) PCT No.: PCT/EP2022/085437

§ 371 Date: Apr. 30, 2024

(30) Foreign Application Priority Data
Dec. 17, 2021 (FR) FR2113832

(51) Int. Cl. A61K 8/42; A61K 8/34; A61K 8/41; A61K 8/49; A61Q 17/04

(52) U.S. Cl. CPC A61K 8/42; A61K 8/345; A61K 8/411; A61K 8/4973; A61Q 17/04

(57) ABSTRACT

The present invention relates to a composition, in particular a cosmetic or dermatological composition, comprising:
a) at least one merocyanine compound corresponding to formula (3) below, and also the geometric isomer forms, in particular E/E or E/Z geometric isomer forms, thereof:

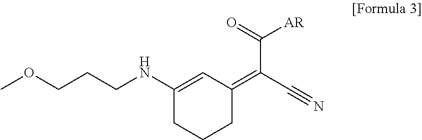

wherein:
A is —O— or —NH—;
R is a C₁-C₂₂ alkyl group, a C₂-C₂₂ alkenyl group, a C₂-C₂₂ alkynyl group, a C₃-C₂₂ cycloalkyl group or a C₃-C₂₂ cycloalkenyl group, it being possible for said groups to be interrupted by one or more O; and
b) dipropylene glycol.

The present invention also relates to a non-therapeutic cosmetic process for caring for and/or making up a keratin material, comprising the application, to the surface of said keratin material, of at least one composition as defined above.

The present invention relates to a cosmetic or dermatological composition comprising at least one merocyanine of formula (3) that will be defined below in detail, and dipropylene glycol.

The present invention also relates to a non-therapeutic cosmetic process for caring for and/or making up a keratin material, comprising the application, to the surface of said keratin material, of at least one composition according to the invention as defined above.

It also relates to a non-therapeutic cosmetic process for limiting the darkening of the skin and/or improving the color and/or the uniformity of the complexion, comprising the application, to the surface of the keratin material, of at least one composition as defined above.

The present invention also relates to a non-therapeutic cosmetic process for preventing and/or treating the signs of aging of a keratin material, comprising the application, to the surface of the keratin material, of at least one composition as defined previously.

It is known that radiation with a wavelength of between 280 nm and 400 nm enables tanning of the human epidermis and that radiation with a wavelength of between 280 and 320 nm, known under the name UVB rays, harms the development of a natural tan. Exposure is also liable to bring about a detrimental change in the biomechanical properties of the epidermis, which is reflected by the appearance of wrinkles, leading to premature ageing of the skin.

It is also known that UVA rays with a wavelength of between 320 and 400 nm penetrate more deeply into the skin than UVB rays. UVA rays cause immediate and persistent tanning of the skin. Daily exposure to UVA rays, even of short duration, under normal conditions can result in damage to the collagen fibers and the elastin, which is reflected by a modification of the microrelief of the skin, the appearance of wrinkles and uneven pigmentation (liver spots, nonuniformity of the complexion).

Protection against UVA and UVB radiation is thus necessary. An effective photoprotective product must protect against both UVA and UVB radiation.

Many photoprotective compositions have been proposed to date to overcome the effects induced by UVA and/or UVB radiation. They generally contain organic UV-screening agents and/or inorganic UV-screening agents, which function according to their own chemical nature and according to their own properties by absorption, reflection or scattering of the UV radiation. They generally contain mixtures of fat-soluble organic screening agents and/or of water-soluble UV-screening agents combined with metal oxide pigments, such as titanium dioxide or zinc oxide.

Many cosmetic compositions intended to limit the darkening of the skin, and to improve the color and uniformity of the complexion have been proposed to date. It is well known in the field of antisun products that such compositions can be obtained by using UV-screening agents and in particular UVB-screening agents. Some compositions can also contain UVA-screening agents. This screening system must cover UVB protection, for the purpose of limiting and controlling the neosynthesis of melanin promoting overall pigmentation, but must also cover UVA protection, in order to limit and control the oxidation of the pre-existing melanin resulting in the darkening of the color of the skin.

However, it is extremely difficult to find a composition containing a particular combination of UV-screening agents which would be especially suitable for the photoprotection of the skin and particularly for an improvement in the quality of the skin both in terms of the color and in terms of its mechanical elasticity properties.

Advantageously, this improvement is particularly desired on skin which is already pigmented, for the purpose of not enhancing either the pigmentary melanin load or the structure of the melanin already present within the skin.

In fact, the majority of organic UV-screening agents are constituted of aromatic compounds which absorb in the range of wavelengths between 280 and 370 nm. In addition to their solar radiation-screening capacity, the desired photoprotective compounds must also have good cosmetic properties, good solubility in the usual solvents and in particular in fatty substances such as oils, and also good photostability alone or in combination with other UV-screening agents. They must also be colorless or at least have a color which is cosmetically acceptable to the consumer.

One of the main disadvantages known to date of these compositions is that these screening systems have insufficient effectiveness against UV radiation and particularly against long UVA radiation with wavelengths above 370 nm with the aim of controlling light-induced pigmentation and the development thereof by a system which screens out UV radiation over the whole of the UV spectrum.

Among all the compounds that have been recommended for this purpose, an advantageous family of UV-screening agents which is constituted of carbonated merocyanine derivatives has been proposed, which is described in patent U.S. Pat. No. 4,195,999, application WO 2004/006878 and document IP COM Journal 4 (4), 16 No. IPCOM000011179D published on Apr. 3, 2004. These compounds exhibit very good screening properties in the long UVA radiation range but exhibit a solubility in the usual solvents, both in the aqueous phase and in the fatty phase, which is only slightly satisfactory and a photostability which is unsatisfactory for some merocyanines.

With the aim of searching for other merocyanines having a better solubility in the usual solvents and a better photostability, merocyanines comprising polar groups constituted of hydroxyl and ether functional groups which show good screening effectiveness in the long UVA radiation range have been proposed in application WO 2013/011094. However, the solubility of these particular merocyanines is still not entirely satisfactory and often requires a tedious formulating process. Moreover, the large amounts of solvent necessary in order to solubilize this type of merocyanine can result in cosmetic unpleasantness, such as a tacky and greasy effect on application.

There thus remains the need to improve the solubility of these merocyanines in cosmetic compositions, in particular photoprotective formulations, both in the aqueous phase and in the fatty phase, while still obtaining good cosmeticity.

The applicant has discovered, surprisingly, that, by using dipropylene glycol, it is possible to substantially improve the solubility of these merocyanines both in an aqueous phase and in a fatty phase. This discovery forms the basis of the present invention.

Thus, in accordance with one of the subjects of the present invention, there is now proposed a cosmetic or dermatological composition comprising at least one merocyanine of formula (3) which will be defined below in detail and dipropylene glycol.

Moreover, there also remains the need to improve the solubility of merocyanines in the presence of organic screening agents. Indeed, the addition of additional screening agents can destabilize compositions comprising a merocyanine.

The applicant has discovered, surprisingly, that, by using dipropylene glycol, it is possible to substantially improve the solubility of these merocyanines in an aqueous phase or in a fatty phase, even in the presence of additional organic UV-screening agents.

The applicant has also discovered that the use of dipropylene glycol makes it possible to obtain good cosmeticity of the composition comprising the merocyanines, said composition being in particular non-greasy and non-tacky.

The present invention also relates to a non-therapeutic cosmetic process for caring for and/or making up a keratin material, comprising the application, to the surface of said keratin material, of at least one composition according to the invention as defined above.

The present invention also relates to a non-therapeutic cosmetic method for limiting the darkening of the skin and/or improving the color and/or the uniformity of the complexion, comprising the application, to the surface of the keratin material, of at least one composition as defined previously.

The present invention also relates to a non-therapeutic cosmetic process for preventing and/or treating the signs of aging of a keratin material, comprising the application, to the surface of the keratin material, of at least one composition as defined previously.

The present invention also relates to the use of dipropylene glycol for solubilizing a merocyanine of formula (3) as defined below, in particular for solubilizing these molecules in the fatty phase and/or in the aqueous phase.

Other features, aspects and advantages of the invention will become apparent on reading the detailed description which follows.

The composition according to the invention is intended for topical application and thus contains a physiologically acceptable medium. The term “physiologically acceptable medium” means here a medium that is compatible with keratin materials.

In the context of the present invention, the term “keratin material” is understood to mean in particular the skin, scalp, keratin fibers, such as the eyelashes, eyebrows, head hair and body hair, nails, mucous membranes, such as the lips, and more particularly the skin and mucous membranes (body, face, area around the eyes, eyelids, lips, preferably body, face and lips).

In the text hereinbelow, unless otherwise indicated, the limits of a range of values are included in that range, notably in the expressions “between” and “ranging from . . . to . . . ”.

Moreover, the expressions “at least one” and “at least” used in the present description are equivalent respectively to the expressions “one or more” and “greater than or equal to”.

According to the invention, the term “preventing” or “prevention” is intended to mean reducing the risk of occurrence or slowing down the occurrence of a given phenomenon, namely, according to the present invention, the signs of aging of a keratin material.

### Merocyanines

According to the present invention, use will be made of a family of merocyanines corresponding to formula (3) below, and also the geometrical isomer forms, notably the E/E or E/Z geometrical isomer forms, thereof:

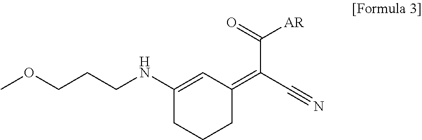

wherein:
A is —O— or —NH—;
R is a C₁-C₂₂ alkyl group, a C₂-C₂₂ alkenyl group, a C₂-C₂₂ alkynyl group, a C₃-C₂₂ cycloalkyl group or a C₃-C₂₂ cycloalkenyl group, it being possible for said groups to be interrupted by one or more O.

The merocyanine compounds of the invention can be in their E/E or E/Z geometrical isomer forms;

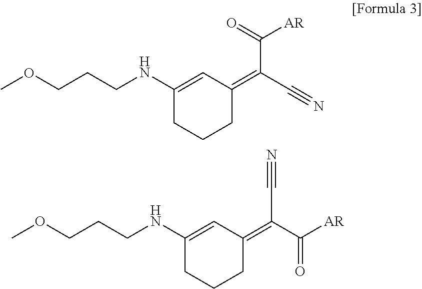

The compounds of formula (3) which are even more preferential are those where:
A is —O—, R is a C₁-C₂₂ alkyl, which can be interrupted by one or more O.

Use will more particularly be made, among the compounds of formula (3), of those chosen from the following group and also their geometrical isomer forms, in particular E/E or E/Z geometrical isomer forms:

TABLE 1

| | |
|---|---|
| 14 | 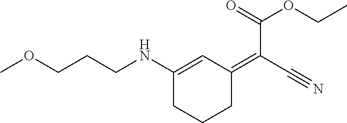 [SMILES: CCOC(=O)/C(C#N)=C1\C=C(NCCCOC)CCC1] |
| | ethyl (2Z)-cyano{3-[(3- |
| | methoxypropyl)amino]cyclohex-2-en- |
| | 1-ylidene}ethanoate |
| 15 | 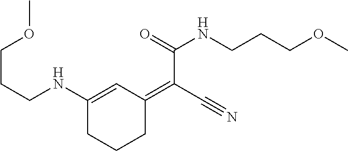 [SMILES: COCCCNC(=O)/C(C#N)=C1\C=C(NCCCOC)CCC1] |
| | (2Z)-2-cyano-N-(3-methoxypropyl)-2- |
| | {3-[(3-methoxypropyl)amino]cyclohex- |
| | 2-en-1-ylidene}ethanamide |
| 25 | 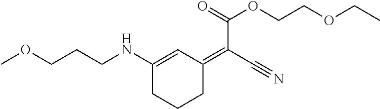 [SMILES: CCOCCOC(=O)/C(C#N)=C1\C=C(NCCCOC)CCC1] |
| | 2-ethoxyethyl (2Z)-cyano{3-[(3- |
| | methoxypropyl)amino]cyclohex-2-en- |
| | 1-ylidene}ethanoate |
| 27 | 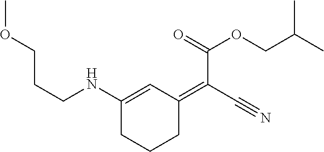 [SMILES: COCCCNC1=C/C(=C(/C#N)C(=O)OCC(C)C)CCC1] |
| | 2-methylpropyl (2Z)-cyano{3-[(3- |
| | methoxypropyl)amino]cyclohex-2-en- |
| | 1-ylidene}ethanoate |
| 29 | 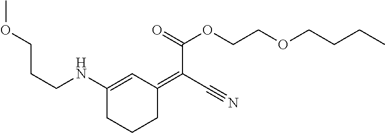 [SMILES: CCCCOCCOC(=O)/C(C#N)=C1\C=C(NCCCOC)CCC1] |
| | 2-butoxyethyl (2Z)-cyano{3-[(3- |
| | methoxypropyl)amino]cyclohex-2-en- |
| | 1-ylidene}ethanoate |
| 31 | 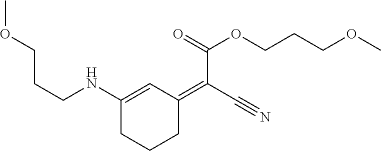 [SMILES: COCCCNC1=C/C(=C(/C#N)C(=O)OCCCOC)CCC1] |
| | 3-methoxypropyl (2Z)-cyano{3-[(3- |
| | methoxypropyl)amino]cyclohex-2-en- |
| | 1-ylidene}ethanoate |
| 37 | 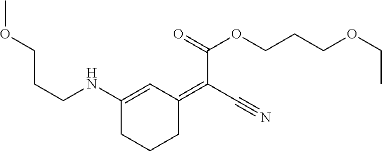 [SMILES: CCOCCCOC(=O)/C(C#N)=C1\C=C(NCCCOC)CCC1] |
| | 3-ethoxypropyl (2Z)-cyano{3-[(3- |
| | methoxypropyl)amino]cyclohex-2-en- |
| | 1-ylidene}ethanoate. |

According to a more particularly preferred form of the invention, use will be made of the compound 2-ethoxyethyl (2Z)-cyano{3-[(3-methoxypropyl)amino]cyclohex-2-en-1-ylidene}ethanoate (25) in its E/E and/or E/Z geometrical configuration.

The E/Z form has the following structure:

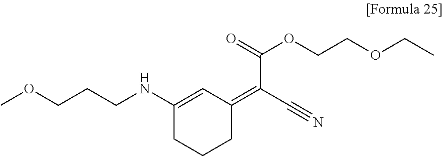 [SMILES: CCOCCOC(=O)/C(C#N)=C1\C=C(NCCCOC)CCC1]

The E/E form has the following structure:

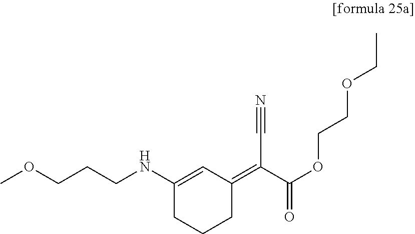 [SMILES: CCOCCOC(=O)/C(C#N)=C1/C=C(NCCCOC)CCC1]

The screening merocyanines in accordance with the invention can be present in the compositions according to the invention in a concentration ranging from 0.1% to 15% by weight, and preferentially from 0.2% to 10% by weight and even better still from 0.5% to 5% by weight, relative to the total weight of the composition.

The compounds of formula (3), which form a carbocyclic ring containing 6 carbon atoms, may be prepared according to the protocols described in patent application WO 2007/071582, in IP.com Journal (2009), 9(5A), 29-30 IPCOM000182396D under the title “Process for producing 3-amino-2-cyclohexan-1-ylidene compounds” and in U.S. Pat. No. 4,749,643 on col. 13, line 66-col. 14, line 57, and the references cited in this regard.

In particular, the compounds of formula (3), such as the compound 2-ethoxyethyl (2Z)-cyano{3-[(3-methoxypropyl)amino]cyclohex-2-en-1-ylidene}ethanoate (25) can be synthesized according to the synthesis scheme described in the publication by B. Winkler et al., Tetrahedron Letters, 55 (2014) 1749-1751, which is entitled “A cyclic merocyanine UV-A absorber: mechanism of formation and crystal structure”, and represented below, for the compounds of formula (3):

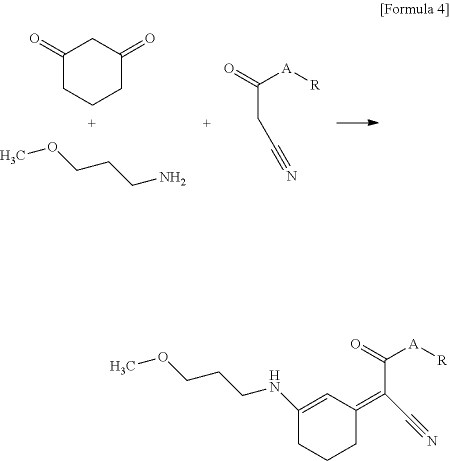

and more particularly for compound 25 described in table 1:

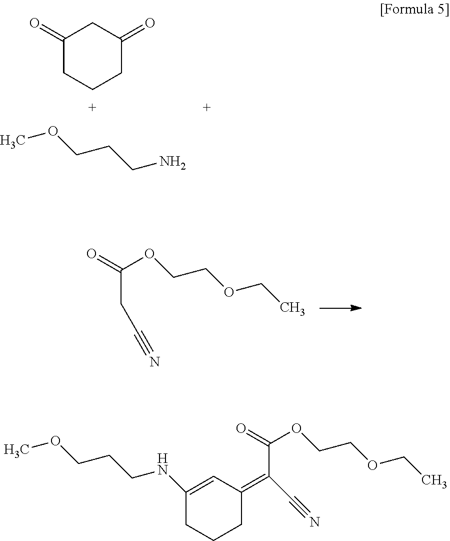

### Dipropylene Glycol

The composition in accordance with the invention comprises dipropylene glycol.

According to one particular embodiment, the dipropylene glycol is present in the composition of the present invention in an amount of between 0.1% and 99%, notably between 0.5% and 50%, preferably between 1% and 25%, and even more preferentially between 1% and 10%, for example between 1% and 5% by weight of the total weight of the composition.

### Additional Acrylic Polymer

According to a particular embodiment of the invention, the composition comprises at least one polymer comprising monomer units of formulae (A) and (B) defined below:

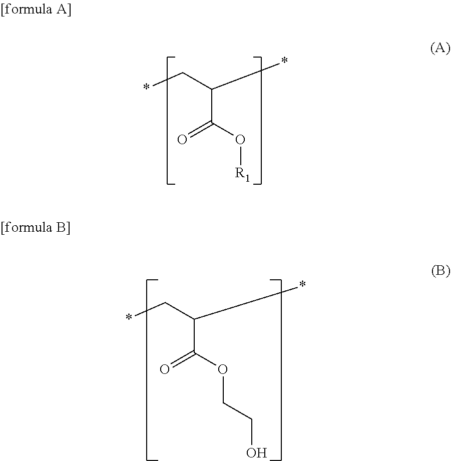

wherein:
R1, independently at each instance, is chosen from alkyl or alkenyl radicals, and
at least 60% by weight of the R₁ groups are radicals chosen from stearyl and behenyl radicals, the percentage by weight relating to the sum of all the R₁ groups present in the polymer, and
the weight ratio of the sum of all the hydroxyethyl acrylate units to the sum of all the acrylate units bearing the R₁ group ranges from 1:30 to 1:1,
and the sum of the total of units A and B is at least 95% by weight relative to the total weight of the polymer.

Preferably, R1 is constituted of alkyl radicals, preferably of C₁₆-C₂₂ alkyl radicals, and more preferentially of stearyl (C₁₈) radicals or of behenyl (C₂₂) radicals.

Preferably, at least 70% by weight of the groups R1 are stearyl or behenyl radicals, preferentially at least 80% by weight and more preferentially at least 90% by weight.

According to one preferred embodiment, all the groups R1 are behenyl radicals.

According to another preferred embodiment, all the R₁ groups are stearyl radicals.

Preferably, said weight ratio ranges from 1:15 to 1:1 and preferentially ranges from 1:10 to 1:4.

Advantageously, the polymer units present in the polymer are constituted of the units (A) and (B) previously described.

The polymer has a number-average molecular weight Mn ranging from 2000 to 9000 g/mol, preferably ranging from 5000 to 9000 g/mol. The number-average molecular weight may be measured via the gel permeation chromatography method, for example according to the method described in the example hereinbelow.

Preferably, the polymer has a melting point ranging from 40° C. to 70° C. and preferentially ranging from 45° C. to 67° C. The melting point is measured by differential scanning calorimetry (DSC), for example according to the method described in the example hereinbelow.

According to a first embodiment, when the polymer is such that at least 60% by weight of the groups R1 are stearyl radicals, then the polymer preferably has a melting point ranging from 40 to 60° C., and preferentially ranging from 45 to 55° C.

According to a second embodiment, when the polymer is such that at least 60% by weight of the groups R1 are behenyl radicals, then the polymer has a melting point ranging from 60° C. to 70° C., and preferentially ranging from 63° C. to 67° C.

The polymer used according to the invention can be prepared by polymerization of a monomer of formula:

CH₂═CH—COO—R₁, R₁ having the meaning previously described, and of 2-hydroxyethyl acrylate.

The polymerization may be performed according to known methods, such as solution polymerization or emulsion polymerization.

The polymerization is, for example, described in document US 2007/0264204.

The acrylic polymer(s) as defined previously can be present in the composition according to the invention in a content of active material ranging from 0.05% to 10% by weight, relative to the total weight of the composition, preferably ranging from 0.1% to 5% by weight and better still ranging from 0.2% to 3% by weight.

### Alkyl or Alkylene Carbonates

According to another particular embodiment, the composition in accordance with the invention comprises at least one alkyl or alkylene carbonate.

According to one preferred embodiment, the alkylene carbonate(s) are chosen notably from those of formula (6) below:

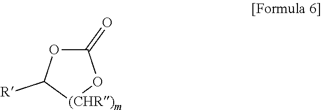

wherein
R′ denotes a hydrogen atom, a linear or branched C₁-C₆ alkyl radical or a linear or branched C₁-C₄ hydroxyalkyl radical;
R″ represents a hydrogen atom, a linear or branched C₁-C₆ alkyl radical or a linear or branched C₁-C₄ hydroxyalkyl radical;
m is 1, 2 or 3.

Preferably, the radical R′ represents a hydrogen atom, a linear or branched C₁-C₄ alkyl radical or a linear or branched C₁-C₂ hydroxyalkyl radical.

R″ represents a hydrogen atom, a linear or branched C₁-C₂ alkyl radical or a linear or branched C₁-C₂ hydroxyalkyl radical.

Preferably, m is 1.

As particularly advantageous examples of alkylene carbonates, mention may be made of the compounds for which the R′ radical represents a hydrogen atom (corresponding to ethylene carbonate), a methyl group (corresponding to propylene carbonate), an ethyl group (corresponding to 1,2-butylene carbonate), a hydroxymethyl group (R′=—CH₂OH; corresponding to glycerol carbonate).

Preferably, the alkylene carbonate used is propylene carbonate.

According to another embodiment, the alkyl carbonate(s) are chosen notably from those of formula (7) below:

R′—O—CO—O—R″ [Formula 7]
wherein:
R′ denotes a linear or branched C₁-C₅ alkyl radical or a linear or branched C₁-C₄ hydroxyalkyl radical;
R″ represents a linear or branched C₁-C₅ alkyl radical or a linear or branched C₁-C₄ hydroxyalkyl radical;
the sum of the carbons of R′ and R″ ranging from 2 to 6.

Preferably, the radical R′ represents a linear C₁-C₃ alkyl radical or a linear C₁-C₂ hydroxyalkyl radical.

R″ represents a linear C₁-C₃ alkyl radical or a linear C₁-C₂ hydroxyalkyl radical.

More particularly, mention may be made of diethyl carbonate and dipropyl carbonate.

The carbonates according to the invention are preferably alkylene carbonates, and more particularly propylene carbonate.

The alkyl or alkylene carbonate(s) are generally present in the compositions according to the invention at concentrations ranging from 0.1% to 98% by weight, preferably from 0.5% to 50% by weight, more preferentially from 1% to 20% by weight and even more particularly from 1% to 10% by weight, for example from 1% to 6% by weight, relative to the total weight of the composition.

### Hydrotrope

According to one particular embodiment of the invention, the composition comprises at least one hydrotrope.

Thus, a composition according to the invention can comprise at least one hydrotrope chosen from nicotinamide, caffeine, salicylic acid salts, the sodium salt of pyroglutamic acid (sodium PCA), sodium 1,3-benzenedisulfonate, sodium benzoate, sodium 4-pyridinecarboxylate, sodium benzenesulfonate, sodium p-toluenesulfonate (NaPTS), sodium butyl monoglycol sulfate (NaBMGS), 4-aminobenzoic acid HCl, sodium cumene sulfonate, N,N-diethyl nicotinamide, N-picolyl nicotinamide, N-allyl nicotinamide, 2-methacryloyloxyethyl phosphorylcholine, resorcinol, pyrogallol, N-picolylacetamide, procaine HCl, proline HCl, pyridine, 3-picolylamine, ibuprofen sodium, sodium xylene sulfonate (SXS), ethyl carbamate, pyridoxal hydrochloride, sodium benzoate, N,N-dimethylacetamide, N-methylacetamide, isoniazid, and mixtures thereof.

According to a preferred embodiment, a composition according to the invention comprises at least one hydrotrope chosen from nicotinamide, caffeine and mixtures thereof. In particular, such a composition may further comprise at least one additional hydrotrope chosen from salicylic acid salts, the sodium salt of pyroglutamic acid (sodium PCA), sodium 1,3-benzenedisulfonate, sodium benzoate, sodium 4-pyridinecarboxylate, sodium benzenesulfonate, sodium p-toluenesulfonate (NaPTS), sodium butyl monoglycol sulfate (NaBMGS), 4-aminobenzoic acid HCl, sodium cumene sulfonate, N,N-diethyl nicotinamide, N-picolyl nicotinamide, N-allyl nicotinamide, 2-methacryloyloxyethyl phosphorylcholine, resorcinol, pyrogallol, N-picolylacetamide, procaine HCl, proline HCl, pyridine, 3-picolylamine, ibuprofen sodium, sodium xylene sulfonate (SXS), ethyl carbamate, pyridoxal hydrochloride, sodium benzoate, N,N-dimethylacetamide, N-methylacetamide, isoniazid, and mixtures thereof.

According to a variant embodiment, a composition according to the invention comprises at least one hydrotrope chosen from nicotinamide, caffeine and mixtures thereof combined with at least one salicylic acid salt.

The salicylic acid salts may in particular be chosen from sodium salicylate, lysine salicylate, arginine salicylate, magnesium salicylate, and mixtures thereof.

Preferably, the salicylic acid salt is sodium salicylate.

According to one preferred embodiment, a composition according to the invention comprises at least nicotinamide.

According to one particular embodiment, a composition according to the invention comprises nicotinamide and sodium salicylate.

When the hydrotrope(s) are present in the composition according to the invention, they are so in an effective amount for solubilizing in water the merocyanine(s) of formula (3) as described above.

The amount of hydrotrope(s) according to the invention present in the compositions according to the invention may range from 0.1% to 20% by weight, in particular from 0.1% to 10% by weight, preferably from 0.5% to 10% by weight, relative to the total weight of the composition.

In particular, the merocyanine(s) of formula (3)/hydrotrope(s) according to the invention weight ratio of a composition according to the invention ranges from 0.1 to 8, preferably from 0.2 to 5, in particular from 0.5 to 3, and more preferentially from 0.8 to 1.5.

According to one particular embodiment, a composition according to the invention comprises at least two different hydrotropes chosen from nicotinamide, caffeine, and sodium salicylate, and the merocyanine(s) of formula (3)/hydrotropes weight ratio of a composition according to the invention ranges from 0.1 to 5, preferably from 0.2 to 3, in particular from 0.4 to 1, and more preferentially from 0.4 to 0.6.

According to one particular embodiment, a composition according to the invention comprises two different hydrotropes, preferably nicotinamide and caffeine, and the weight ratio between these two hydrotropes ranges from 0.2 to 5, in particular from 0.5 to 3, and more preferentially from 0.8 to 1.2.

### Fatty Phase

The composition in accordance with the invention can comprise at least one fatty phase.

For the purposes of the invention, the term “fatty phase” is understood to mean a phase comprising at least one fatty substance, which is notably liquid, solid or pasty, and all of the liposoluble and lipophilic ingredients used for the formulation of the compositions of the invention.

According to one particular embodiment, the composition according to the invention comprises at least one oil.

The term “oil” is intended to mean any fatty substance that is in liquid form at ambient temperature (20-25° C.) and atmospheric pressure (760 mmHg).

The fatty phase can comprise notably at least one volatile or nonvolatile hydrocarbon oil and/or one volatile and/or nonvolatile silicone oil and/or one volatile and/or nonvolatile fluoro oil.

For the purposes of the present invention, the term “silicone oil” is understood to mean an oil comprising at least one silicon atom and in particular at least one Si—O group.

The term “hydrocarbon oil” is understood to mean an oil mainly containing hydrogen and carbon atoms and optionally one or more heteroatoms, in particular nitrogen and oxygen. Thus, these oils may in particular contain one or more carboxyl, ester, ether, hydroxyl functions.

The term “fluoro oil” refers to an oil comprising at least one fluorine atom.

For the purposes of the invention, the term “volatile oil” is intended to mean an oil which is capable of evaporating on contact with the skin or the keratin fiber in less than one hour, at ambient temperature and atmospheric pressure. The volatile oil(s) of the invention are volatile cosmetic oils, which are liquid at ambient temperature, having a non-zero vapor pressure, at ambient temperature and atmospheric pressure, ranging in particular from 0.13 Pa to 40 000 Pa (10-3 to 300 mmHg), in particular ranging from 1.3 Pa to 13 000 Pa (0.01 to 100 mmHg) and more particularly ranging from 1.3 Pa to 1300 Pa (0.01 to 10 mmHg).

The term “nonvolatile oil” is intended to mean an oil that remains on the skin or the keratin fiber at ambient temperature and atmospheric pressure for at least several hours, and that notably has a vapor pressure of less than 10-3 mmHg (0.13 Pa).

### Hydrocarbon Oils

The composition in accordance with the invention can comprise at least one ester of C₂-C₂₂ di- or tricarboxylic acid and of C₁-C₂₄ alcohols.

The C₂-C₂₂ di- or tricarboxylic acids are in particular chosen from citric acid, malic acid, malonic acid, succinic acid, adipic acid, maleic acid, fumaric acid, tartaric acid, isocitric acid, and mixtures thereof. The acids are preferably citric acid and adipic acid, and even more preferentially the acid is citric acid.

The C₁-C₂₄ alcohols are not oxyalkylenated. They may be aliphatic, cyclic or aromatic, having from 1 to 24 carbon atoms. They are in particular chosen from phenol, benzyl alcohol, lauryl alcohol, myristyl alcohol, cetyl alcohol, stearyl alcohol, methanol, ethanol, propanol, n-butanol, t-butanol, pentanol and hexanol. Preferably, the alcohol(s) is/are C₁-C₆ alcohol(s) and can be chosen from methanol, ethanol, propanol, n-butanol, t-butanol, pentanol, hexanol, and even more preferentially the alcohol is ethanol.

The ester(s) of C₂-C₂₂ di- or tricarboxylic acid and of C₁-C₂₄ alcohols can be mono- or polyesterified.

Monoesterified is understood to mean that only one of the two or three carboxylic acid functions is esterified. Polyesterified is understood to mean that at least two carboxylic acid functions are esterified.

The di- or tricarboxylic acid can be esterified with several different alcohols. It is preferably esterified with just one alcohol.

According to a particular embodiment of the invention, the composition comprises at least one ester of C₃-C₂₂ tricarboxylic acid and of C₁-C₂₄, preferably C₁-C₆, alcohols. This/these ester(s) can be mono-, di- or triesterified.

Monoesterified is understood to mean that only one of the three carboxylic acid functions is esterified. Diesterified is understood to mean that two of the three carboxylic acid functions are esterified. Triesterified is understood to mean that all three carboxylic acid functions are esterified.

According to a particular embodiment of the invention, the ester(s) of C₃-C₂₂ tricarboxylic acid and of C₁-C₆ alcohols are triesterified.

According to a particular embodiment of the invention, the ester(s) of C₃-C₂₂ tricarboxylic acid and of C₁-C₆ alcohols are chosen from the compounds of formula (8) below:

(R₁O—CO)CH₂—C(R)(OC—OR₂)—CH₂(OC—OR₃)
wherein:
R₁, R₂ and R₃ represent, independently of each other, a hydrogen atom or a monovalent, saturated or unsaturated, aliphatic, cyclic or aromatic hydrocarbon group having from 1 to 6 carbon atoms,
R represents a hydrogen atom or a hydroxyl radical.

According to a preferred embodiment, R₁, R₂ and R₃ represent, independently of each other, a hydrogen atom or a linear or branched, substituted or unsubstituted, preferably unsubstituted, C₁-C₆ alkyl radical, and in particular a radical chosen from methyl, ethyl, propyl, n-butyl, t-butyl, pentyl and hexyl radicals. Preferably, R₁, R₂ and R₃ are chosen, independently of each other, from a hydrogen atom and methyl, ethyl, propyl, n-butyl and t-butyl radicals.

According to a preferred embodiment of the invention, the radicals R₁, R₂ and R₃ are identical and are chosen from C₁-C₆, preferably C₁-C₄, alkyl radicals and even more preferentially are ethyl radicals.

According to a preferred embodiment of the invention, R represents a hydroxyl radical.

According to a particular embodiment of the invention, the ester of tricarboxylic acid and of C₁-C₆ alcohols has the following formula:

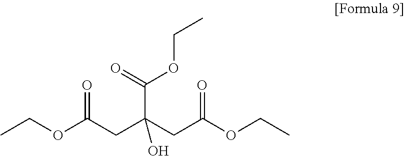 [SMILES: CCOC(=O)CC(O)(CC(=O)OCC)C(=O)OCC]

As an example of an ester of C₃-C₂₂ tricarboxylic acid and of C₁-C₆ alcohols of formula (10) and having the INCI name Triethyl Citrate, mention will be made of the product sold under the name Citrofol AI Extra by Jungbunzlauer.

Mention may also be made, as nonvolatile hydrocarbon oils which can be used according to the invention, of:
(i) hydrocarbon oils of plant origin, such as glyceride triesters, which are generally triesters of fatty acids and of glycerol, the fatty acids of which can have varied chain lengths from C₄ to C₂₄, it being possible for these chains to be saturated or unsaturated and linear or branched; these oils are in particular wheat germ oil, sunflower oil, grape seed oil, sesame oil, maize oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, red kuri squash oil, pumpkin oil, blackcurrant oil, evening primrose oil, millet oil, barley oil, quinoa oil, rye oil, safflower oil, candlenut oil, passionflower oil or musk rose oil; or alternatively triglycerides of caprylic/capric acids, such as those sold by Stearinerie Dubois or those sold under the names Miglyol 810®, 812® and 818® by Dynamit Nobel;
(ii) synthetic ethers having from 10 to 40 carbon atoms;
(iii) linear or branched hydrocarbons of mineral or synthetic origin, such as liquid petroleum, polydecenes, hydrogenated polyisobutene, such as Parleam, squalane and mixtures thereof;
(iv) synthetic esters, such as the oils of formula RCOOR′ wherein R represents the residue of a linear or branched fatty acid comprising from 1 to 40 carbon atoms and R′ represents a hydrocarbon chain, in particular branched hydrocarbon chain, containing from 1 to 40 carbon atoms, with the proviso that R+R′≥10, such as, for example, Purcellin oil (cetostearyl octanoate), isopropyl myristate, isopropyl palmitate, C₁₂-C₁₅ alkyl benzoate, such as the product sold under the trade name Finsolv TN® or Witconol TN® by Witco or Tegosoft TN® by Evonik Goldschmidt, 2-ethylphenyl benzoate, such as the commercial product sold under the name X-Tend 226® by ISP, isopropyl lanolate, hexyl laurate, diisopropyl adipate, isononyl isononanoate, oleyl erucate, 2-ethylhexyl palmitate, isostearyl isostearate, diisopropyl sebacate, such as the product sold under the name Dub Dis by Stearinerie Dubois, octanoates, decanoates or ricinoleates of alcohols or of polyalcohols, such as propylene glycol dioctanoate; hydroxylated esters, such as isostearyl lactate or diisostearyl malate; and pentaerythritol esters; citrates or tartrates, such as di(linear C₁₂-C₁₃ alkyl) tartrates, such as those sold under the name Cosmacol ETI® by Enichem Augusta Industriale, and also di(linear C₁₄-C₁₅ alkyl) tartrates, such as those sold under the name Cosmacol ETL® by the same company; acetates;
(v) fatty alcohols which are liquid at ambient temperature, comprising a branched and/or unsaturated carbon-based chain having from 12 to 26 carbon atoms, such as octyldodecanol, isostearyl alcohol, oleyl alcohol, 2-hexyldecanol, 2-butyloctanol or 2-undecylpentadecanol;
(vi) higher C₁₂-C₂₂ fatty acids, such as oleic acid, linoleic acid or linolenic acid;
(vii) carbonates, such as dicaprylyl carbonate, such as the product sold under the name Cetiol CCO by Cognis;
and mixtures thereof.

Among the nonvolatile hydrocarbon oils which can be used according to the invention, preference will be given more particularly to glyceride triesters and in particular to caprylic/capric acid triglycerides, synthetic esters and in particular diisopropyl adipate, diisopropyl sebacate, isopropyl palmitate, dicaprylyl carbonate, isononyl isononanoate, oleyl erucate, C₁₂-C₁₅ alkyl benzoate, 2-ethylphenyl benzoate and fatty alcohols, in particular octyldodecanol. Preferably, the nonvolatile hydrocarbon oils are chosen from diisopropyl adipate, diisopropyl sebacate, isopropyl palmitate and dicaprylyl carbonate.

As volatile hydrocarbon oils which can be used according to the invention, mention may be made in particular of hydrocarbon oils containing from 8 to 16 carbon atoms and in particular of branched C₈-C₁₆ alkanes, such as C₈-C₁₆ isoalkanes of petroleum origin (also known as isoparaffins), such as isododecane (also known as 2,2,4,4,6-pentamethylheptane), isodecane or isohexadecane, the oils sold under the Isopar or Permethyl trade names, branched C₈-C₁₆ esters, isohexyl neopentanoate, and mixtures thereof.

Mention may also be made of the alkanes described in the Cognis patent applications WO 2007/068 371 or WO 2008/155 059 (mixtures of different alkanes differing by at least one carbon). These alkanes are obtained from fatty alcohols, which are themselves obtained from coconut kernel or palm oil. Mention may be made of the mixtures of n-undecane (C₁₁) and n-tridecane (C₁₃) obtained in Examples 1 and 2 of application WO 2008/155 059 from Cognis. Mention may also be made of n-dodecane (C₁₂) and n-tetradecane (C₁₄) sold by Sasol respectively under the references Parafol 12-97 and Parafol 14-97®, and also mixtures thereof.

Other volatile hydrocarbon oils, such as petroleum distillates, in particular those sold under the name Shell Solt® by Shell, can also be used. According to one embodiment, the volatile solvent is chosen from volatile hydrocarbon oils having from 8 to 16 carbon atoms, and mixtures thereof.

### Silicone Oils

The nonvolatile silicone oils may be notably chosen from nonvolatile polydimethylsiloxanes (PDMSs), polydimethylsiloxanes including alkyl or alkoxy groups which are pendent and/or at the end of the silicone chain, these groups each containing from 2 to 24 carbon atoms, or phenyl silicones, such as phenyl trimethicones, phenyl dimethicones, phenyltrimethylsiloxydiphenylsiloxanes, diphenyl dimethicones, diphenylmethyldiphenyltrisiloxanes or 2-phenylethyl trimethylsiloxysilicates.

Mention may be made, as volatile silicone oils, for example, of volatile linear or cyclic silicone oils, in particular those having a viscosity 8 centistokes (8×10⁻⁶ m²/s) and having in particular from 2 to 7 silicon atoms, these silicones optionally comprising alkyl or alkoxy groups having from 1 to 10 carbon atoms. Mention may in particular be made, as volatile silicone oil which can be used in the invention, of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane and mixtures thereof.

A fatty phase according to the invention can additionally comprise other fatty substances, mixed with or dissolved in the oil.

Another fatty substance which can be present in the fatty phase can, for example, be:
a fatty acid chosen from fatty acids including from 8 to 30 carbon atoms, such as stearic acid, lauric acid, palmitic acid and oleic acid;
a wax chosen from waxes such as lanolin, beeswax, carnauba or candelilla wax, rice bran wax, paraffin waxes, lignite waxes, microcrystalline waxes, ceresin or ozokerite, or synthetic waxes, such as polyethylene waxes or Fischer-Tropsch waxes;
a gum chosen from silicone gums (dimethiconol);
a pasty compound, such as polymeric or non-polymeric silicone compounds, esters of an oligomeric glycerol, arachidyl propionate, fatty acid triglycerides and their derivatives;
and mixtures thereof.

Preferentially, the overall fatty phase, including all the lipophilic substances other than lipophilic screening agents of the composition capable of being dissolved in this same phase, represents from 5% to 95% by weight and preferentially from 10% to 80% by weight, relative to the total weight of the composition.

### Aqueous Phase

The composition in accordance with the invention can comprise at least one aqueous phase.

The aqueous phase contains water and optionally other water-soluble or water-miscible organic solvents.

An aqueous phase which is suitable for the invention can comprise, for example, a water chosen from a natural spring water, such as water from La Roche-Posay, water from Vittel, water from Saint-Gervais Mont-Blanc or waters from Vichy, or a floral water.

According to one specific form of the invention, the overall aqueous phase, including all the hydrophilic substances other than hydrophilic screening agents of the composition capable of being dissolved in this same phase, represents from 1% to 99% by weight and preferentially from 10% to 80% by weight, relative to the total weight of the composition.

### Additives

### Additional UV-Screening Agents

The compositions according to the invention may also contain one or more additional UV-screening agents chosen from hydrophilic, lipophilic or insoluble organic UV-screening agents and/or one or more mineral pigments. It will preferentially be constituted of at least one hydrophilic, lipophilic or insoluble organic UV-screening agent.

The term “hydrophilic UV-screening agent” is intended to mean any cosmetic or dermatological organic or inorganic compound for screening out UV radiation, which can be fully dissolved in molecular form in a liquid aqueous phase or else which can be dissolved in colloidal form (for example in micellar form) in a liquid aqueous phase.

The term “lipophilic screening agent” is intended to mean any cosmetic or dermatological organic or inorganic compound for screening out UV radiation, which can be fully dissolved in molecular form in a liquid fatty phase or else which can be dissolved in colloidal form (for example in micellar form) in a liquid fatty phase.

The term “insoluble UV-screening agent” is intended to mean any cosmetic or dermatological organic or inorganic compound for screening out UV radiation which has a solubility in water of less than 0.5% by weight and a solubility of less than 0.5% by weight in the majority of organic solvents such as liquid paraffin, fatty alcohol benzoates and fatty acid triglycerides, for example Miglyol 812® sold by Dynamit Nobel. This solubility, determined at 70° C., is defined as the amount of product in solution in the solvent at equilibrium with an excess of solid in suspension after returning to ambient temperature. It can be easily evaluated in the laboratory.

The additional organic UV-screening agents are chosen in particular from cinnamic compounds; anthranilate compounds; salicylic compounds; dibenzoylmethane compounds; benzylidenecamphor compounds; benzophenone compounds; β,β-diphenylacrylate compounds; triazine compounds; benzotriazole compounds; benzalmalonate compounds, in particular those cited in patent U.S. Pat. No. 5,624,663; benzimidazole derivatives; imidazoline compounds; bis-benzazolyl compounds, such as described in patents EP 669 323 and U.S. Pat. No. 2,463,264; p-aminobenzoic (PABA) compounds; methylenebis(hydroxyphenylbenzotriazole) compounds, such as described in applications U.S. Pat. Nos. 5,237,071, 5,166,355, GB 2 303 549, DE 197 26 184 and EP 893 119; benzoxazole compounds, such as described in patent applications EP 0 832 642, EP 1 027 883, EP 1 300 137 and DE 101 62 844; screening polymers and screening silicones, such as those described in particular in application WO 93/04665; dimers derived from α-alkylstyrene, such as those described in patent application DE 198 55 649; 4,4-diarylbutadiene compounds, such as described in applications EP 0 967 200, DE 197 46 654, DE 197 55 649, EP-A-1 008 586, EP 1 133 980 and EP 133 981, and mixtures thereof.

Mention may be made, as examples of organic photoprotective agents, of those denoted below under their INCI names.

### Cinnamic Compounds:

Ethylhexyl Methoxycinnamate, sold in particular under the trade name Parsol MCX® by DSM Nutritional Products,
Isopropyl Methoxycinnamate,
Isoamyl p-Methoxycinnamate, sold under the trade name Neo Heliopan E 1000® by Symrise,
DEA Methoxycinnamate,
Diisopropyl Methylcinnamate,
Glyceryl Ethylhexanoate Dimethoxycinnamate.

### Dibenzoylmethane Compounds:

Butylmethoxydibenzoylmethane, sold in particular under the trade name Parsol 1789® by DSM Nutritional Products, Isopropyl Dibenzoylmethane.

### Para-Aminobenzoic Compounds:

PABA,
Ethyl PABA,
Ethyl Dihydroxypropyl PABA,
Ethylhexyl Dimethyl PABA, sold in particular under the name Escalol 507® by ISP,
Glyceryl PABA,
PEG-25 PABA, sold under the name Uvinul P 25® by BASF.

### Salicylic Compounds:

Homosalate, sold under the name Eusolex HMS® by Rona/EM Industries,
Ethylhexyl Salicylate, sold under the name Neo Heliopan OS® by Symrise,
Dipropylene Glycol Salicylate, sold under the name Dipsal® by Scher,
TEA Salicylate, sold under the name Neo Heliopan TS® by Symrise.

### β,β-Diphenylacrylate Compounds:

Octocrylene, sold in particular under the trade name Uvinul N 539® by BASF,
Etocrylene, sold in particular under the trade name Uvinul N 35® by BASF.

### Benzophenone Compounds:

Benzophenone-1, sold under the trade name Uvinul 400® by BASF,
Benzophenone-2, sold under the trade name Uvinul D 50® by BASF,
Benzophenone-3 or Oxybenzone, sold under the trade name Uvinul M 40® by BASF,
Benzophenone-4, sold under the trade name Uvinul MS 40® by BASF,
Benzophenone-5,
Benzophenone-6, sold under the trade name Helisorb 11® by Norquay,
Benzophenone-8, sold under the trade name Spectra-Sorb UV-24® by American Cyanamid,
Benzophenone-9, sold under the trade name Uvinul DS 49® by BASF,
Benzophenone-12,
n-Hexyl 2-(4-diethylamino-2-hydroxybenzoyl)benzoate, sold under the trade name Uvinul A Plus® or, as a mixture with octyl methoxycinnamate, under the trade name Uvinul A Plus B® by BASF,
1,1′-(1,4-Piperazinediyl)bis[1-[2-[4-(diethylamino)-2-hydroxybenzoyl]phenyl]methanone] (CAS 919803-06-8), as described in application WO 2007/071584; this compound advantageously being used in micronized form (mean size of 0.02 to 2 μm), which may be obtained, for example,
according to the micronization process described in applications GB-A-2 303 549 and EP-A-893 119, and in particular in the form of an aqueous dispersion.

### Benzylidenecamphor Compounds:

3-Benzylidene Camphor, manufactured under the name Mexoryl SD® by Chimex,
4-Methylbenzylidene Camphor, sold under the name Eusolex 6300® by Merck,
Benzylidenecamphorsulfonic acid, manufactured under the name Mexoryl SL® by Chimex,
Camphor benzalkonium methosulfate, manufactured under the name Mexoryl SO® by Chimex,
Terephthalylidenedicamphorsulfonic acid, manufactured under the name Mexoryl SX® by Chimex,
Polyacrylamidomethylbenzylidenecamphor, manufactured under the name Mexoryl SW® by Chimex.

Phenylbenzimidazole compounds:
Phenylbenzimidazolesulfonic acid, sold in particular under the trade name Eusolex 232® by Merck.

### Bisbenzoazolyl Compounds:

Disodium phenyl dibenzimidazole tetrasulfonate sold under the trade name Neo Heliopan AP® by Haarmann and Reimer.

### Phenylbenzotriazole Compounds:

Drometrizole Trisiloxane, sold under the name Silatrizole® by Rhodia Chimie.

### Methylenebis(Hydroxyphenylbenzotriazole) Compounds:

Methylene Bis-Benzotriazolyl Tetramethylbutylphenol, in particular in solid form, such as the product sold under the trade name Mixxim BB/100® by Fairmount Chemical, or in the form of an aqueous dispersion of micronized particles having a mean particle size which varies from 0.01 to 5 μm, more preferentially from 0.01 to 2 μm and more particularly from 0.020 to 2 μm, with at least one alkylpolyglycoside surfactant having the structure CₙH₂ₙ₊₁O(C₆H₁₀O₅)ₓH, wherein n is an integer from 8 to 16 and x is the mean degree of polymerization of the (C₆H₁₀O₅) unit and varies from 1.4 to 1.6, such as described in patent GB-A-2 303 549, sold in particular under the trade name Tinosorb M® by BASF, or in the form of an aqueous dispersion of micronized particles having a mean particle size which varies from 0.02 to 2 μm, more preferentially from 0.01 to 1.5 μm and more particularly from 0.02 to 1 μm, in the presence of at least one polyglyceryl mono(C₈-C₂₀)alkyl ester having a degree of glycerol polymerization of at least 5, such as the aqueous dispersions described in application WO 2009/063392.

Triazine compounds:
3,3′-(1,4-Phenylene)bis(5,6-diphenyl-1,2,4-triazine), with the INCI name Phenylene Bis-Diphenyltriazine, and with the following chemical structure:

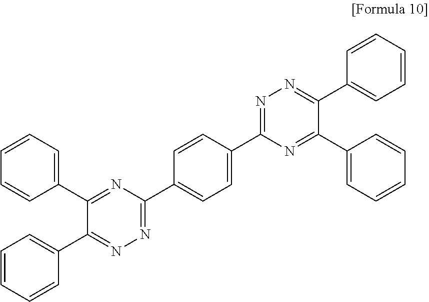 [SMILES: c1ccc(-c2nnc(-c3ccc(-c4nnc(-c5ccccc5)c(-c5ccccc5)n4)cc3)nc2-c2ccccc2)cc1]

Bis-Ethylhexyloxyphenol Methoxyphenyl Triazine, sold under the trade name Tinosorb S® by BASF,
Ethylhexyl Triazone, sold in particular under the trade name Uvinul T 150® by BASF,
Diethylhexyl Butamido Triazone, sold under the trade name Uvasorb HEB® by Sigma 3V,
2,4,6-tris(dineopentyl 4′-aminobenzalmalonate)-s-triazine,
2,4,6-tris(diisobutyl 4′-aminobenzalmalonate)-s-triazine,
2,4-bis(n-butyl 4′-aminobenzoate)-6-(aminopropyltrisiloxane)-s-triazine,
2,4-bis(dineopentyl 4′-aminobenzalmalonate)-6-(n-butyl 4′-aminobenzoate)-s-triazine,
symmetrical triazine screening agents substituted by naphthalenyl groups or polyphenyl groups described in patent U.S. Pat. No. 6,225,467, application WO2004/085412 (see compounds 6 and 9) or the document “Symmetrical Triazine Derivatives”, IP.COM IPCOM000031257 Journal, INC West Henrietta, NY, US (20 Sep. 2004), in particular 2,4,6-tris(diphenyl)triazine and 2,4,6-tris(terphenyl)triazine, which is also mentioned in patent applications WO06/035000, WO06/034982, WO06/034991, WO06/035007, WO2006/034992 and WO2006/034985, these compounds advantageously being used in micronized form (mean particle size of 0.02 to 3 μm), which can be obtained, for example, according to the micronization process described in applications GB-A-2 303 549 and EP-A-893 119, and in particular in aqueous dispersion form,
silicone triazines substituted by two aminobenzoate groups, such as described in patent EP 0 841 341, in particular 2,4-bis(n-butyl 4′-aminobenzalmalonate)-6-[(3-{1,3,3,3-tetramethyl-1-[(trimethylsilyl)oxy]disiloxanyl}propyl)amino]-s-triazine.

### Anthranilic Compounds:

Menthyl anthranilate, sold under the trade name Neo Heliopan MA® by Symrise.

### Imidazoline Compounds:

Ethylhexyl Dimethoxybenzylidene Dioxoimidazoline Propionate.

### Benzalmalonate Compounds:

Polyorganosiloxane comprising benzalmalonate functional groups, such as Polysilicone-15, sold under the trade name Parsol SLX® by Hoffmann-La Roche.

### 4,4-Diarylbutadiene Compounds:

1,1-Dicarboxy(2,2′-dimethylpropyl)-4,4-diphenylbutadiene.

### Benzoxazole Compounds:

2,4-Bis[5-(1-dimethylpropyl)benzoxazol-2-yl(4-phenyl)imino]-6-(2-ethylhexyl)imino-1,3,5-triazine, sold under the name Uvasorb K2A® by Sigma 3V.

The preferential organic screening agents are chosen from:
Ethylhexyl Methoxycinnamate,
Ethylhexyl Salicylate,
Homosalate,
Butyl Methoxydibenzoylmethane,
Octocrylene,
Phenylbenzimidazole Sulfonic Acid,
Benzophenone-3,
Benzophenone-4,
Benzophenone-5,
n-Hexyl 2-(4-diethylamino-2-hydroxybenzoyl)benzoate,
4-Methylbenzylidene Camphor,
Terephthalylidene Dicamphor Sulfonic Acid,
Disodium Phenyl Dibenzimidazole Tetrasulfonate,
Methylene Bis-Benzotriazolyl Tetramethylbutylphenol,
Bis-Ethylhexyloxyphenol Methoxyphenyl Triazine,
Ethylhexyl Triazone,
Diethylhexyl Butamido Triazone,
2,4,6-Tris(dineopentyl 4′-aminobenzalmalonate)-s-triazine,
2,4,6-Tris(diisobutyl 4′-aminobenzalmalonate)-s-triazine,
2,4-Bis(n-butyl 4′-aminobenzoate)-6-(aminopropyltrisiloxane)-s-triazine,
2,4-Bis(dineopentyl 4′-aminobenzalmalonate)-6-(n-butyl 4′-aminobenzoate)-s-triazine,
2,4-Bis(n-butyl 4′-aminobenzalmalonate)-6-[(3-{1,3,3,3-tetramethyl-1-[(trimethylsilyl)oxy]disiloxanyl}propyl)amino]-s-triazine,
2,4,6-Tris(diphenyl)triazine,
2,4,6-Tris(terphenyl)triazine,
Drometrizole Trisiloxane,
Polysilicone-15,
1,1-Dicarboxy(2,2′-dimethylpropyl)-4,4-diphenylbutadiene,
2,4-Bis[4-[5-(1,1-dimethylpropyl)benzoxazol-2-yl]phenylimino]-6-[(2-ethylhexyl)imino]-1,3,5-triazine,
and mixtures thereof.

The particularly preferred organic screening agents are chosen from:
Ethylhexyl Salicylate,
Homosalate,
Butyl Methoxydibenzoylmethane,
Octocrylene,
n-Hexyl 2-(4-diethylamino-2-hydroxybenzoyl)benzoate,
Terephthalylidene Dicamphor Sulfonic Acid,
Bis-Ethylhexyloxyphenol Methoxyphenyl Triazine,
Ethylhexyl Triazone,
Diethylhexyl Butamido Triazone,
2,4-Bis(n-butyl 4′-aminobenzalmalonate)-6-[(3-{1,3,3,3-tetramethyl-1-[(trimethylsilyl)oxy]disiloxanyl}propyl)amino]-s-triazine,
Drometrizole Trisiloxane,
and mixtures thereof.

The inorganic UV-screening agents used in accordance with the present invention are metal oxide pigments. More preferentially, the inorganic UV-screening agents of the invention are metal oxide particles having a mean elementary particle size of less than or equal to 0.5 μm, more preferentially of between 0.005 and 0.5 μm, more preferentially still of between 0.01 and 0.2 μm, better still between 0.01 and 0.1 μm and more particularly between 0.015 and 0.05 μm.

They may be chosen in particular from titanium oxide, zinc oxide, iron oxide, zirconium oxide and cerium oxide, or mixtures thereof.

Such coated or uncoated metal oxide pigments are described in particular in patent application EP-A-0 518 773. Commercial pigments that may be mentioned include the products sold by the companies Sachtleben Pigments, Tayca, Merck and Degussa.

The metal oxide pigments may be coated or uncoated.

The coated pigments are pigments which have undergone one or more surface treatments of chemical, electronic, mechanochemical and/or mechanical nature with compounds such as amino acids, beeswax, fatty acids, fatty alcohols, anionic surfactants, lecithins, sodium, potassium, zinc, iron or aluminum salts of fatty acids, metal alkoxides (of titanium or aluminum), polyethylene, silicones, proteins (collagen, elastin), alkanolamines, silicon oxides, metal oxides or sodium hexametaphosphate.

The coated pigments are more particularly titanium oxides coated:
with silica, such as the product Sunveil® from Ikeda,
with silica and iron oxide, such as the product Sunveil F® from Ikeda,
with silica and alumina, such as the products Microtitanium Dioxide MT 500 SA® and Microtitanium Dioxide MT 100 SA from Tayca and Tioveil from Tioxide,
with alumina, such as the products Tipaque TTO-55 (B)® and Tipaque TTO-55 (A)® from Ishihara and UVT 14/4 from Sachtleben Pigments,
with alumina and aluminum stearate, such as the products Microtitanium Dioxide MT 100 T®, MT 100 TX®, MT 100 Z® and MT-01® from Tayca, the products Solaveil CT-10 W® and Solaveil CT 100® from Uniqema and the product Eusolex T-AVO® from Merck,
with silica, alumina and alginic acid, such as the product MT-100 AQ® from Tayca,
with alumina and aluminum laurate, such as the product Microtitanium Dioxide MT 100 S® from Tayca,
with iron oxide and iron stearate, such as the product Microtitanium Dioxide MT 100 F® from Tayca,
with zinc oxide and zinc stearate, such as the product BR 351® from Tayca,
with silica and alumina and treated with a silicone, such as the products Microtitanium Dioxide MT 600 SAS®, Microtitanium Dioxide MT 500 SAS® or Microtitanium Dioxide MT 100 SAS® from Tayca,
with silica, alumina and aluminum stearate and treated with a silicone, such as the product STT-30-DS® from Titan Kogyo,
with silica and treated with a silicone, such as the product UV-Titan X 195® from Sachtleben Pigments,
with alumina and treated with a silicone, such as the products Tipaque TTO-55 (S)® from Ishihara or UV Titan M 262® from Sachtleben Pigments,
with triethanolamine, such as the product STT-65-S from Titan Kogyo,
with stearic acid, such as the product Tipaque TTO-55 (C)® from Ishihara,
with sodium hexametaphosphate, such as the product Microtitanium Dioxide MT 150 W® from Tayca,
TiO₂ treated with octyltrimethylsilane, sold under the trade name T 805® by Degussa Silices,
TiO₂ treated with a polydimethylsiloxane, sold under the trade name 70250 Cardre UF TiO₂SI3® by Cardre,
anatase/rutile TiO₂ treated with a polydimethylhydrosiloxane, sold under the trade name Micro Titanium Dioxide USP Grade Hydrophobic® by Color Techniques,
TiO₂ coated with triethylhexanoin, aluminum stearate and alumina, sold under the trade name Solaveil CT-200-LQ-(WD) by Croda,
TiO₂ coated with aluminum stearate, with alumina and with silicone sold under the trade name Solaveil CT-12W-LQ-(WD) by Croda,
TiO₂ coated with lauroyl lysine sold by Daito Kasei Kogyo under the name LL 5 Titanium Dioxyde CR 50,
TiO₂ coated with C9-15 fluoroalcohol phosphate and with aluminum hydroxide sold by Daito Kasei Kogyo under the name PFX-5 TiO₂ CR-50.

Mention may also be made of TiO₂ pigments doped with at least one transition metal such as iron, zinc or manganese and more particularly manganese. Preferably, said doped pigments are in the form of an oily dispersion. The oil present in the oily dispersion is preferably chosen from triglycerides, including those of capric/caprylic acids. The oily dispersion of titanium oxide particles can additionally comprise one or more dispersing agents, such as, for example, a sorbitan ester, such as sorbitan isostearate, a polyoxyalkylenated glycerol fatty acid ester, such as Tri-PPG-3 Myristyl Ether Citrate and Polyglyceryl-3 Polyricinoleate. Preferably, the oily dispersion of titanium oxide particles comprises at least one dispersing agent chosen from polyoxyalkylenated glycerol fatty acid esters. Mention may more particularly be made of the oily dispersion of TiO₂ particles doped with manganese in capric/caprylic acid triglyceride in the presence of Tri-PPG-3 Myristyl Ether Citrate and Polyglyceryl-3 Polyricinoleate and Sorbitan Isostearate with the INCI name: titanium dioxide (and) TRI-PPG-3 myristyl ether citrate (and) polyglyceryl-3 ricinoleate (and) sorbitan isostearate, for instance the product sold under the trade name Optisol TD50® by Croda.

The uncoated titanium oxide pigments are sold, for example, by Tayca under the trade names Microtitanium Dioxide MT 500 B or Microtitanium Dioxide MT 600 B®, by Degussa under the name P 25, by Wackher under the name Transparent titanium oxide PW®, by Miyoshi Kasei under the name UFTR®, by Tomen under the name ITS® and by Tioxide under the name Tioveil AQ.

The uncoated zinc oxide pigments are, for example:
those sold under the name Z-Cote by Sunsmart;
those sold under the name Nanox® by Elementis;
those sold under the name Nanogard WCD 2025® by Nanophase Technologies.

The coated zinc oxide pigments are, for example:
those sold under the name Zinc Oxide CS-5® by Toshibi (ZnO coated with polymethylhydrosiloxane);
those sold under the name Nanogard Zinc Oxide FN® by Nanophase Technologies (as a 40% dispersion in Finsolv TN®, C₁₂-C₁₅ alkyl benzoates);
those sold under the name Daitopersion Zn-30® and Daitopersion Zn-50® by Daito (dispersions in oxyethylenated cyclopolymethylsiloxane/polydimethylsiloxane, containing 30% or 50% of zinc oxides coated with silica and polymethylhydrosiloxane);
those sold under the name NFD Ultrafine ZnO® by Daikin (ZnO coated with perfluoroalkyl phosphate and copolymer based on perfluoroalkylethyl in dispersion in cyclopentasiloxane);
those sold under the name SPD-Z1® by Shin-Etsu (ZnO coated with silicone-grafted acrylic polymer, dispersed in cyclodimethylsiloxane);
those sold under the name Escalol Z100® by ISP (alumina-treated ZnO dispersed in the ethylhexyl methoxycinnamate/PVP-hexadecene copolymer/methicone mixture);
those sold under the name Fuji ZnO-SMS-10® by Fuji Pigment (ZnO coated with silica and polymethylsilsesquioxane);
those sold under the name Nanox Gel TN® by Elementis (ZnO dispersed at 55% in C₁₂-C₁₅ alkyl benzoates with hydroxystearic acid polycondensate).

The uncoated cerium oxide pigments may be, for example, those sold under the name Colloidal Cerium Oxide® by Rhone-Poulenc.

The uncoated iron oxide pigments are, for example, sold by Arnaud under the names Nanogard WCD 2002® (FE 45B®), Nanogard Iron FE 45 BL AQ, Nanogard FE 45R AQ® and Nanogard WCD 2006® (FE 45R®) or by Mitsubishi under the name TY-220®.

The coated iron oxide pigments are, for example, sold by Arnaud under the names Nanogard WCD 2008 (FE 45B FN)®, Nanogard WCD 2009® (FE 45B 556®), Nanogard FE 45 BL 345® and Nanogard FE 45 BL® or by BASF under the name Transparent Iron Oxide®.

Mention may also be made of mixtures of metal oxides, in particular of titanium dioxide and cerium dioxide, including the mixture in equal weights of titanium dioxide and cerium dioxide coated with silica, sold by Ikeda under the name Sunveil A®, and also the mixture of titanium dioxide and zinc dioxide coated with alumina, silica and silicone, such as the product M 261® sold by Sachtleben Pigments, or coated with alumina, silica and glycerol, such as the product M 211® sold by Sachtleben Pigments.

According to the invention, coated or uncoated titanium oxide pigments are particularly preferred.

The additional UV-screening agents according to the invention can be present in the composition according to the invention in a content ranging from 0.1% to 60% by weight and in particular from 5% to 30% by weight relative to the total weight of the composition.

### Other Additives

The composition in accordance with the present invention may also comprise conventional cosmetic adjuvants chosen in particular from organic solvents, ionic or nonionic thickeners, softeners, humectants, opacifiers, stabilizers, emollients, silicones, antifoams, fragrances, preserving agents, anionic, cationic, nonionic, zwitterionic or amphoteric surfactants, active agents, fillers, polymers, propellants, basifying or acidifying agents or any other ingredient commonly used in the cosmetic and/or dermatological field.

Among the organic solvents, mention may be made of short-chain monoalcohols, for example C₁-C₄ monoalcohols, such as ethanol and isopropanol, short-chain C₂-C₈ polyols, such as glycerol or diols, such as caprylyl glycol, 1,2-pentanediol, propanediol, butanediol, glycols and glycol ethers, such as ethylene glycol, propylene glycol, butylene glycol, hexylene glycol, dipropylene glycol or diethylene glycol, 2-ethoxyethanol, diethylene glycol monomethyl ether, triethylene glycol monomethyl ether, sorbitol, and mixtures thereof.

According to a preferred embodiment, use may more particularly be made of ethanol, propylene glycol, glycerol, and mixtures thereof.

Mention may be made, as thickeners, of carboxyvinyl polymers, such as the Carbopols® (Carbomers) and the Pemulens, such as Pemulen TR1® and Pemulen TR2® (acrylate/C₁₀-C₃₀ alkyl acrylate copolymer); polyacrylamides, for instance the crosslinked copolymers sold under the names Sepigel 305® (CTFA name: polyacrylamide/C₁₃₋₁₄ isoparaffin/Laureth 7) or Simulgel 600 (CTFA name: acrylamide/sodium acryloyldimethyl taurate copolymer/isohexadecane/polysorbate 80) by SEPPIC; 2-acrylamido-2-methylpropanesulfonic acid polymers and copolymers, optionally crosslinked and/or neutralized, such as the poly(2-acrylamido-2-methylpropanesulfonic acid) sold by Hoechst under the trade name Hostacerin AMPS® (CTFA name: ammonium polyacryloyldimethyl taurate) or Simulgel 800®, sold by SEPPIC (CTFA name: sodium polyacryloyldimethyl taurate/polysorbate 80/sorbitan oleate); copolymers of 2-acrylamido-2-methylpropanesulfonic acid and of hydroxyethyl acrylate, such as Simulgel NS® and Sepinov EMT 10®, sold by SEPPIC; cellulose derivatives, such as hydroxyethylcellulose; polysaccharides and in particular gums, such as xanthan gum; water-soluble or water-dispersible silicone derivatives, such as acrylic silicones, polyether silicones and cationic silicones, and mixtures thereof.

Among the acidifying agents, examples that may be mentioned include mineral or organic acids, for instance hydrochloric acid, orthophosphoric acid, sulfuric acid, carboxylic acids, for instance acetic acid, tartaric acid, citric acid or lactic acid, and sulfonic acids.

Among the basifying agents, examples that may be mentioned include aqueous ammonia, alkali metal carbonates, alkanolamines, such as mono-, di- and triethanolamines and derivatives thereof, sodium hydroxide or potassium hydroxide.

Preferably, the cosmetic composition comprises one or more basifying agents chosen from alkanolamines, in particular triethanolamine, and sodium hydroxide.

Among the active agents for caring for keratin materials such as the skin, the lips, the scalp, the hair, the eyelashes or the nails, mention may be made for example of vitamins and derivatives or precursors thereof, alone or as mixtures; antioxidants; free-radical scavengers; anti-pollutants; self-tanning agents; anti-glycation agents; calmatives; deodorant agents; essential oils; NO-synthase inhibitors; agents for stimulating the synthesis of dermal or epidermal macromolecules and/or for preventing degradation thereof; agents for stimulating fibroblast proliferation; agents for stimulating keratinocyte proliferation; muscle relaxants; refreshing agents; tensioning agents; mattifying agents; depigmenting agents; propigmenting agents; keratolytic agents; desquamating agents; moisturizing agents; anti-inflammatories; antimicrobials; thinning agents; agents which act on cell energy metabolism; insect repellents; substance P antagonists or CRGP antagonists; agents for preventing hair loss; antiwrinkle agents; anti-aging agents.

Those skilled in the art will select said active agent(s) as a function of the effect desired on the skin, the hair, the eyelashes, the eyebrows and the nails.

Needless to say, those skilled in the art will take care to select the abovementioned optional additional compound(s) and/or the amounts thereof so that the advantageous properties intrinsically associated with the compositions in accordance with the invention are not, or not substantially, adversely affected by the envisaged addition(s).

### Presentation Forms

The compositions in accordance with the invention may be aqueous or anhydrous.

When the compositions are aqueous, they contain at least one aqueous phase.

They can then be in purely aqueous form, that is to say they comprise an amount of fatty phase of less than 10% by weight, preferably less than 5% by weight and even more preferentially less than 2% by weight, relative to the total weight of the composition. Advantageously, the composition in accordance with the invention is essentially aqueous, i.e. it does not contain a fatty phase.

The compositions according to the invention may also be in particular in the form of a simple or complex (O/W, W/O, O/W/O or W/O/W) emulsion, such as a cream, a milk or a gel-cream.

In the case where the composition in accordance with the invention is aqueous, and it is possible to measure its pH, this pH is generally between 3 and 12 approximately, preferably between 5 and 9 approximately, and even more particularly from 5.5 to 8.

The compositions may also be in anhydrous form, such as for example in the form of an oil, an alcoholic solution or a glycolic solution. The term “anhydrous composition” is intended to mean a composition containing less than 1% by weight of water, or even less than 0.5% water, and especially free of water, the water not being added during the preparation of the composition but corresponding to the residual water provided by the mixed ingredients. They can optionally be packaged as an aerosol and be provided in the form of a foam or of a spray.

In the case of compositions in the form of oil-in-water or water-in-oil emulsions, the emulsification processes which can be used are of the paddle or propeller, rotor-stator and HPH type.

In order to obtain stable emulsions with a low content of polymer (oil/polymer ratio>25), it is possible to prepare the dispersion in concentrated phase and then to dilute the dispersion with the remainder of the aqueous phase.

It is also possible, via HPH (between 50 and 800 bar), to obtain stable dispersions with drop sizes that can be as small as 100 nm.

The emulsions generally contain at least one emulsifier chosen from amphoteric, anionic, cationic or nonionic emulsifiers, used alone or as a mixture. The emulsifiers are appropriately chosen according to the emulsion to be obtained (W/O or O/W).

Mention may be made, as examples of W/O emulsifying surfactants, of alkyl esters or ethers of sorbitan, of glycerol, of polyol or of sugars; or silicone surfactants, such as dimethicone copolyols, for example the mixture of cyclomethicone and dimethicone copolyol sold under the name DC 5225 C® by Dow Corning, and alkyl dimethicone copolyols, such as lauryl methicone copolyol, sold under the name Dow Corning 5200 Formulation Aid by Dow Corning, or cetyl dimethicone copolyol, such as the product sold under the name Abil EM 90R® by Goldschmidt and the mixture of cetyl dimethicone copolyol, polyglyceryl isostearate (4 mol) and hexyl laurate sold under the name Abil WE 09® by Goldschmidt. One or more coemulsifiers, which may be chosen advantageously from the group comprising polyol alkyl esters, may also be added thereto.

Mention may also be made of non-silicone emulsifying surfactants, in particular alkyl esters or ethers of sorbitan, of glycerol, of polyol or of sugars.

Polyol alkyl esters that may in particular be mentioned include polyethylene glycol esters, for instance PEG-30 dipolyhydroxystearate, such as the product sold under the name Arlacel P135® by ICI.

Mention may be made, as glycerol and/or sorbitan esters, for example, of polyglyceryl isostearate, such as the product sold under the name Isolan GI 34® by Goldschmidt; sorbitan isostearate, such as the product sold under the name Arlacel 987® by IC; sorbitan glyceryl isostearate, such as the product sold under the name Arlacel 986® by ICI, and mixtures thereof.

Mention may be made, for the O/W emulsions, for example, as nonionic emulsifying surfactants, of polyoxyalkylenated (more particularly polyoxyethylenated and/or polyoxypropylenated) esters of fatty acids and of glycerol; oxyalkylenated esters of fatty acids and of sorbitan; polyoxyalkylenated (in particular polyoxyethylenated and/or polyoxypropylenated) esters of fatty acids, optionally in combination with an ester of a fatty acid and of glycerol, such as the PEG-100 Stearate/Glyceryl Stearate mixture sold, for example, by ICI under the name Arlacel 165; oxyalkylenated (oxyethylenated and/or oxypropylenated) ethers of fatty alcohols; esters of sugars, such as sucrose stearate; or ethers of fatty alcohol and of sugar, in particular alkyl polyglucosides (APGs), such as decyl glucoside and lauryl glucoside, sold, for example, by Henkel under the respective names Plantaren 2000® and Plantaren 1200®, cetostearyl glucoside, optionally as a mixture with cetostearyl alcohol, sold, for example, under the name Montanov68® by SEPPIC, under the name Tegocare CG90® by Goldschmidt and under the name Emulgade KE3302® by Henkel, and arachidyl glucoside, for example in the form of the mixture of arachidyl and behenyl alcohols and of arachidyl glucoside sold under the name Montanov 202® by SEPPIC. According to one particular embodiment of the invention, the mixture of the alkylpolyglucoside as defined above with the corresponding fatty alcohol may be in the form of a self-emulsifying composition, for example as described in WO-A-92/06778.

When it is an emulsion, the aqueous phase of this emulsion may comprise a nonionic vesicular dispersion prepared according to known processes (Bangham, Standish and Watkins, J. Mol. Biol., 13, 238 (1965), FR 2 315 991 and FR 2 416 008).

The compositions according to the invention have applications in a large number of treatments, in particular cosmetic treatments, of the skin, lips and hair, including the scalp, in particular for protecting and/or caring for the skin, lips and/or hair and/or for making up the skin and/or lips.

Another subject of the present invention is constituted of the use of the compositions according to the invention as defined above in the manufacture of products for the cosmetic treatment of the skin, lips, nails, hair, eyelashes, eyebrows and/or scalp, in particular of care products, sun protection products and makeup products.

The cosmetic compositions according to the invention can be used, for example, as makeup products.

Another subject of the present invention is constituted of a non-therapeutic cosmetic process for caring for and/or making up a keratin material, which consists in applying, to the surface of said keratin material, at least one composition according to the invention as defined above.

Another subject of the invention is constituted of the use of dipropylene glycol to solubilize a merocyanine of formula (3) as defined above.

According to one particular embodiment, the dipropylene glycol makes it possible to solubilize the merocyanines in accordance with the invention in the fatty phase and/or in the aqueous phase.

The cosmetic compositions according to the invention can, for example, be used as care products and/or sun protection products for the face and/or body with a liquid to semi-liquid consistency, such as milks, more or less smooth creams, gel-creams or pastes. They can optionally be packaged as an aerosol and be provided in the form of a foam or of a spray.

The compositions according to the invention in the form of vaporizable fluid lotions in accordance with the invention are applied to the skin or the hair in the form of fine particles by means of pressurization devices. The devices in accordance with the invention are well known to those skilled in the art and comprise non-aerosol pumps or “atomizers”, aerosol containers comprising a propellant and aerosol pumps using compressed air as propellant. These devices are described in patents U.S. Pat. Nos. 4,077,441 and 4,850,517.

The compositions packaged as an aerosol in accordance with the invention generally contain conventional propellants, such as, for example, hydrofluorinated compounds, dichlorodifluoromethane, difluoroethane, dimethyl ether, isobutane, n-butane, propane or trichlorofluoromethane. They are preferably present in amounts ranging from 15% to 50% by weight, relative to the total weight of the composition.

### Assembly

According to another aspect, the invention also relates to a cosmetic assembly comprising:
i) a container delimiting one or more compartment(s), said container being closed by a closing member and optionally being unsealed, and
ii) a makeup and/or care composition in accordance with the invention placed inside said compartment(s).

The container may be, for example, in the form of a jar or a box.

The closing member may be in the form of a lid comprising a cap mounted so as to be able to move by translation or by pivoting relative to the container housing said makeup and/or care composition(s).

### EXAMPLES

The examples which follow serve to illustrate the invention without, however, exhibiting a limiting nature.

### Example A1: Preparation of the Compound (14)

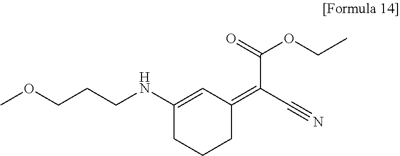 [SMILES: CCOC(=O)/C(C#N)=C1\C=C(NCCCOC)CCC1]

122.23 grams of 3-[(3-methoxypropyl)amino]-2-cyclohexen-1-one are alkylated with dimethyl sulfate or alternatively with diethyl sulfate and treated with 75.45 grams of ethyl cyanoacetate in approximately equimolar proportions in the presence of a base and optionally of a solvent.

The base/solvent combinations indicated in the following table are used.

TABLE 2

| Example | Base | Solvent |
|---|---|---|
| Example A1.1 | DBU (1,8- | dimethylacetamide |
| | diazabicyclo[5.4.0]undec- | |
| | 7-ene) | |
| Example A1.2 | triethylamine | isopropanol |
| Example A1.3 | 3-methoxypropylamine | isopropanol |
| Example A1.4 | 3-methoxypropylamine | tert-amyl alcohol |
| Example A1.5 | 3-methoxypropylamine | toluene |
| Example A1.6 | 3-methoxypropylamine | dimethylformamide |
| Example A1.7 | 3-methoxypropylamine | without solvent |
| Example A1.8 | N-morpholine | isopropanol |

The completion of the alkylation reaction can be monitored, for example, by methods such as TLC, GC or HPLC.

162.30 grams of compound (14) are obtained in the form of a brown oil.

After crystallization, the product is obtained in the form of yellowish crystals.
Melting point: 92.7° C.

### Example A2: Preparation of the Compound (15)

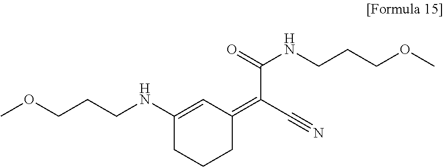

101.00 grams of 3-[(3-methoxypropyl)amino]-2-cyclohexen-1-one are alkylated with dimethyl sulfate or alternatively with diethyl sulfate and treated with 86.00 grams of 2-cyano-N-(3-methoxypropyl)acetamide in approximately equimolar proportions in the presence of a base and optionally of a solvent.

The base/solvent combinations indicated in the following table are used.

TABLE 3

| Example | Base | Solvent |
|---|---|---|
| Example A2.1 | DBU (1,8- | dimethylacetamide |
| | diazabicyclo[5.4.0]undec- | |
| | 7-ene) | |
| Example A2.2 | triethylamine | isopropanol |
| Example A2.3 | 3-methoxypropylamine | isopropanol |
| Example A2.4 | 3-methoxypropylamine | tert-amyl alcohol |
| Example A2.5 | 3-methoxypropylamine | toluene |
| Example A2.6 | 3-methoxypropylamine | dimethylformamide |
| Example A2.7 | 3-methoxypropylamine | without solvent |

The crude product (15) is obtained in the form of a dark brown oil.

After silica gel column chromatography (eluent: 99/1 toluene/methanol), 81.8 grams of product are obtained in the form of yellowish crystals.
Melting point: 84.7-85.3° C.

### Example A3: Preparation of the Compound (27)

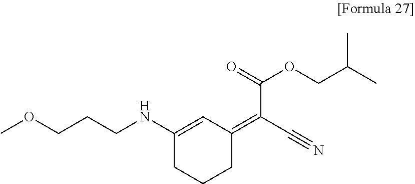 [SMILES: COCCCNC1=C/C(=C(/C#N)C(=O)OCC(C)C)CCC1]

13.09 grams of 3-[(3-methoxypropyl)amino]-2-cyclohexen-1-one are alkylated with dimethyl sulfate or alternatively with diethyl sulfate and treated with 10.12 grams of isobutyl cyanoacetate in the presence of a base and optionally of a solvent.

The base/solvent combinations indicated in the following table are used.

TABLE 4

| Example | Base | Solvent |
|---|---|---|
| Example A3.1 | DBU (1,8- | dimethylacetamide |
| | diazabicyclo[5.4.0]undec- | |
| | 7-ene) | |
| Example A3.2 | triethylamine | isopropanol |
| Example A3.3 | 3-methoxypropylamine | isopropanol |
| Example A3.4 | N-methylmorpholine | tert-amyl alcohol |
| Example A3.5 | 3-methoxypropylamine | toluene |
| Example A3.6 | 3-methoxypropylamine | dimethylformamide |
| Example A3.7 | 3-methoxypropylamine | without solvent |

15.97 grams of crude product (27) are obtained in the form of a dark brown oil.

After silica gel column chromatography (eluent: toluene/acetone), 13.46 grams of product are obtained in the form of yellowish crystals.
Melting point: 96.3° C.

### Example A4: Preparation of the Compound (25)

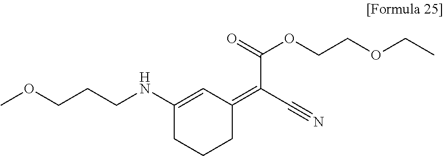 [SMILES: CCOCCOC(=O)/C(C#N)=C1\C=C(NCCCOC)CCC1]

148.4 grams of 3-[(3-methoxypropyl)amino]-2-cyclohexen-1-one are alkylated with dimethyl sulfate or alternatively with diethyl sulfate and treated with 130.00 grams of 2-ethoxyethyl cyanoacetate in the presence of an organic base and of a solvent.

The base/solvent combinations indicated in the table below are used.

TABLE 5

| Example | Base | Solvent |
|---|---|---|
| Example A4.1 | DBU (1,8- | dimethylacetamide |
| | diazabicyclo[5.4.0]undec- | |
| | 7-ene) | |
| Example A4.2 | triethylamine | isopropanol |
| Example A4.3 | 3-methoxypropylamine | isopropanol |
| Example A4.4 | N-methylmorpholine | tert-amyl alcohol |
| Example A4.5 | 3-methoxypropylamine | toluene |
| Example A4.6 | 3-methoxypropylamine | dimethylformamide |
| Example A4.7 | 3-methoxypropylamine | without solvent |

### Example of Preparation of Acrylic Polymers:

### Determination of the Molecular Weight by Gel Permeation Chromatography (GPC):

The sample is prepared by preparing a solution of the polymer at 10 mg/ml in tetrahydrofuran. The sample is placed in an oven at 54° C. for 10 minutes and then in an oscillating shaker for 60 minutes to aid dissolution. After visual inspection, the sample appears to be totally dissolved in the solvent.

The sample prepared was analyzed using two polypore 300×7.5 mm columns (manufactured by Agilent Technologies), a Waters 2695 chromatographic system, a tetrahydrofuran mobile phase and detection by refractive index. The sample was filtered through a 0.45 μm nylon filter, before being injected into the liquid chromatograph. The standards used for the calibration are the Easi Vial narrow polystyrene (PS) standards from Agilent Technologies.

Polystyrene standards ranging from 2 520 000 to 162 daltons were used for the calibration.

The system is equipped with a PSS SECcurity 1260 RI detector. The polystyrene calibration curve was used to determine the average molecular weight. The recording of the diagrams and the determination of the various molecular weights were performed by the Win GPC Unichrom 81 program.

### Determination of the Melting Point by Differential Scanning Calorimetry (or DSC):

This method describes the general procedure for determining the melting point of polymers by differential scanning calorimetry. This method is based on standards ASTM E791 and ASTM D 34182 and the DSC calibration is performed according to standard ASTM E 9672.

### Behenyl Acrylate/2-Hydroxyethyl Acrylate Copolymer (Polymer 1):

In a 4-necked flask equipped with a side-blade mixer, an internal thermometer, two funnels, a reflux condenser, and an extension for two other necks, 175 g of behenyl acrylate, 25 g of 2-hydroxyethyl acrylate and 0.4 g of 2,2′-azobis(2-methylbutyronitrile) (Akzo Nobel) were added, over the course of 60 minutes at 80° C., to 40 g of isopropanol, with stirring, after having removed the oxygen from the system by means of a nitrogen purge for 20 minutes. The mixture was stirred at 80° C. for 3 hours. The solvent was then removed by vacuum distillation, 1 g of dilauryl peroxide was then added and the reaction was continued for 60 minutes at 110° C. The step was repeated. The mixture was then cooled to 90° C., a stream of demineralized water was added and the mixture was then stirred. The water was removed by vacuum distillation.
Molecular weight: Mn=7300 g/mol, Mw=21 000, Mw/Mn=2.8
Melting point: 65° C.

### Stearyl Acrylate/2-Hydroxyethyl Acrylate Copolymer (Polymer 2):

In a 4-necked flask equipped with a side-blade mixer, an internal thermometer, two funnels, a reflux condenser, and an extension for two other necks, 155 g of stearyl acrylate, 45 g of 2-hydroxyethyl acrylate and 0.4 g of 2,2′-azobis(2-methylbutyronitrile) (Akzo Nobel) were added, over the course of 90 minutes at 80° C., to 50 g of isopropanol, with stirring, after having removed the oxygen from the system by means of a nitrogen flush for 20 minutes. The mixture was stirred at 80° C. for 3 hours. The solvent was then removed by vacuum distillation, 1 g of dilauryl peroxide was then added and the reaction was continued for 60 minutes at 125° C. The step was repeated. The mixture was then cooled to 90° C., a stream of demineralized water was added and the mixture was then stirred. The water was removed by vacuum distillation.
Molecular weight: Mn=7500 g/mol, Mw=19 000, Mw/Mn=2.6
Melting point: 49° C.

### Formulation Examples

In these examples, the amounts of the composition ingredients are given as % by weight of starting materials, relative to the total weight of the composition.

### Protocol for Evaluating Solubility

The solubility of merocyanine in the aqueous and/or oily solutions can be evaluated macroscopically and/or microscopically. It is considered that the merocyanine is soluble if, at ambient temperature, the solution appears to the eye to be clear and translucent, and it does not have any visible crystals under a white-light or polarized-light microscope (objective ×20 to ×40).

In the examples that follow, the solubility is evaluated macroscopically. It is evaluated at ambient temperature, on the day the solution is prepared and then over time. During this time period, the solutions are stored at ambient temperature.

### Comparative Examples 1 to 3

The following solutions were prepared according to the process below.

TABLE 6

| | 1 | 2 | |
|---|---|---|---|
| | (outside the | (outside the | 3 |
| Ingredients | invention) | invention) | (invention) |
| Compound (25) | 5 | 5 | 5 |
| Glycerol | 95 | — | — |
| Propylene glycol | — | 95 | — |
| Dipropylene glycol | — | — | 95 |
| Solubility at t₀ | insoluble | soluble | soluble |
| Solubility at 24 hours at | — | insoluble | soluble |
| ambient temperature | | | |
| Solubility at 1 month at | | insoluble | soluble |
| ambient temperature | | | |

### Preparation Method for Compositions 1 to 3

The various raw materials are successively introduced into a container before being mixed using a magnetic stirrer and being heated to 80° C. to 90° C. for 10 minutes to 1 hour, until all the compounds are solubilized.

The mixture is then then left to stand in order to return to ambient temperature.

### Results

The previous tests show that dipropylene glycol significantly improves the solubility of merocyanines.

### Comparative Examples 4 and 5—Aqueous Serum

The following composition was prepared according to the process below.

TABLE 7

| | | Example 4 | |
|---|---|---|---|
| | | (outside the | Example 5 |
| Phase | Ingredients | invention) | (invention) |
| A1 | Propanediol | 3 | 3 |
| A1 | Chelating agent | 0.3 | 0.3 |
| A1 | Caprylyl glycol | 0.3 | 0.3 |
| A1 | Propylene carbonate | 20 | 20 |
| A1 | Dipropylene glycol | — | 20 |
| A1 | PEG-8 - Polyethylene glycol (8 EO) | 20 | — |
| A1 | Methoxypropylamino | 3 | 3 |
| | cyclohexenylidene | | |
| | ethoxyethylcyanoacetate | | |
| | (Compound 25) | | |
| A2 | AQUA | 48.4 | 48.4 |
| B | Denat. alcohol | 5 | 5 |

### Preparation Method:

All the ingredients of phase A1 are mixed and the water of phase A2 is slowly added, while heating to 50° C. and stirring with a magnetic bar. Once the mixture is clear, it is left to return to ambient temperature and the ethanol is introduced.

The mixture is stored at ambient temperature for 2 months.

The solubility state is determined macroscopically with the naked eye and checked under an optical microscope.

### Results

After two months at ambient temperature, crystallization of compound 25 is observed in composition 4, which is not the case in composition 5.

These comparative examples show that the presence of dipropylene glycol in the composition makes it possible to improve the solubility of compound 25.

### Example 6—Aqueous Serum

The following composition was prepared according to the process below.

TABLE 8

| | | Example 6 |
|---|---|---|
| Phase | Ingredients | (invention) |
| A1 | Caprylyl Glycol - Octane-1,2-diol | 0.3 |
| A1 | Propanediol - Propane-1,3-diol | 3 |
| A1 | Polysorbate 20 - Oxyethylenated (20 EO) sorbitan | 1 |
| | monolaurate | |
| A1 | PEG-8 - Polyethylene glycol (8 EO) | 10 |
| A1 | Dipropylene glycol | 10 |
| A1 | Methoxypropylamino cyclohexenylidene | 3 |
| | ethoxyethylcyanoacetate (Compound 25) | |
| A2 | AQUA | 37 |
| A2 | Terephthalylidene Dicamphor Sulfonic Acid | 12 |
| | (Mexoryl SX from Noveal) | |
| A2 | Phenylbenzimidazole Sulfonic Acid (Eusolex 232 | 8 |
| | from Merck) | |
| A2 | pH adjuster | qs pH 6.8 |
| B | Denat. alcohol | 10 |

### Preparation Method:

All the ingredients of phase A1 are mixed while heating to 50° C. and with magnetic stirring until a clear mixture is obtained. Phase A2 is prepared by heating to 50° C. and stirring until a clear mixture is obtained. Phase A2 is slowly added to phase A1. Once the mixture is clear, it is left to return to ambient temperature and the ethanol is introduced.

### Example 7—Cream

TABLE 9

| Phase | Ingredients | Example 7 |
|---|---|---|
| A1 | AQUA | 41.6 |
| A1 | Methoxypropylamino cyclohexenylidene | 1.5 |
| | ethoxyethylcyanoacetate (Compound 25) | |
| A1 | PEG-8 - Polyethylene glycol (8 EO) | 4 |
| A1 | Dipropylene glycol | 4 |
| A1 | Preserving agent(s) | 0.5 |
| A1 | Chelating agent | 0.3 |
| A2 | Acrylates copolymer (Carbopol Aqua SF-1 - Lubrizol) | 2 |
| A3 | Triethanolamine | 0.3 |
| B1 | Butyl Methoxydibenzoylmethane (Parsol 1789 from | 2 |
| | DSM Nutritional Products) | |
| B1 | Ethylhexyl Salicylate (Parsol EHS from DSM | 5 |
| | Nutritional Products) | |
| B1 | Ethylhexyl Triazone (Uvinul T150 from BASF) | 4.5 |
| B1 | Drometrizole Trisiloxane (Mexoryl XL from Noveal) | 2.5 |
| B1 | Diethylamino Hydroxybenzoyl Hexyl Benzoate | 1 |
| | (Uvinul A Plus Granular from BASF) | |
| B1 | Bis-Ethylhexyloxyphenol Methoxyphenyl Triazine | 3 |
| | (Tinosorb S from BASF) | |
| B1 | Caprylyl Glycol - Octane-1,2-diol | 0.3 |
| B1 | Diisopropyl Sebacate | 8 |
| B1 | Diisopropyl adipate | 4 |
| B1 | C12-22 Alkyl Acrylate/Hydroxyethyl Acrylate | 2.5 |
| | Copolymer (Polymer 1) | |
| B2 | Titanium Dioxide (MT100 TV from Tayca) | 2.5 |
| C1 | Acrylates/C10-30 Alkyl Acrylate Crosspolymer | 0.15 |
| | (Pemulen TR-1 Polymer from Lubrizol) | |
| C1 | Sodium Polyacrylate (Cosmedia SP from BASF) | 0.2 |
| C2 | Triethanolamine | 0.15 |
| D | SILICA | 1 |
| D | Aluminum Starch Octenylsuccinate | 2 |
| E | Denat. Alcohol | 7 |

### Preparation Method:

Phase A1 is prepared by mixing all the ingredients until a clear phase is obtained. Phase A2 is dispersed and then phase A3 is added to neutralize the gel and enable the development thereof. All this preparation of the phase, which will be referred to hereinafter as phase A, is carried out by heating to 65° C. and stirring with blades (50 rpm).

Phase B1 is prepared by mixing all the ingredients by heating to 80° C. with magnetic stirring until a clear and homogeneous phase is obtained. Phase B2 is then dispersed in B1. This phase will be referred to as phase B.

The emulsification is carried out by dispersing phase B in phase A at 65° C. and with vigorous stirring (blades at 80 rpm and turbine at 11 000 rpm) for 5 minutes. An oil-in-water emulsion is obtained. The temperature of the mixture is then gradually reduced to return to ambient temperature while maintaining the same stirring until the end. Around 55° C., the thickeners of phase C1 are added (5 minutes), then neutralized with C2 for development of the gels (5 minutes). The fillers of phase D are dispersed (5 minutes) from 35° C. Finally, the alcohol of phase E is added once the mixture has returned to ambient temperature.

### Example 8

The following composition is prepared according to the process described in detail below.

TABLE 10

| | | 8 |
|---|---|---|
| Phase | Ingredients | (invention) |
| A1 | Compound (25) | 3% |
| A1 | Propylene carbonate (Merck) | 5% |
| A2 | Glycerol | 6% |
| A2 | Dipropylene glycol (CABB Group GmbH) | 5% |
| A3 | Terephthalylidene dicamphor sulfonic acid | 0.9% |
| | (Mexoryl SX from Noveal) | |
| A3 | Triethanolamine | 0.15% |
| A3 | Nicotinamide (ThermoFisher) | 2.5% |
| A3 | Sodium Salicylate (Sigma Aldrich) | 0.5% |
| A3 | Water | q.s. 70 |
| A4 | Ethanol (Sigma Aldrich) | 4% |

| | |
|---|---|
| Solubility at t₀ | soluble |
| Solubility at 4° C. after 4 weeks | soluble |

### Procedure:

Compound 25 is solubilized in propylene carbonate at 65° C. with magnetic stirring (phase A1). Next, the glycerol and dipropylene glycol are added (phase A2), then the solution containing phase A3 is introduced. Phase A3 was prepared beforehand by mixing all the ingredients until solubilized. Finally, the ethanol is added with gentle stirring after returning to ambient temperature.

The samples are kept at 4° C. for 4 weeks. The solubility is evaluated macroscopically by eye and microscopically under an optical microscope.

### Example 9

The following composition is prepared according to the process as described in detail below.

TABLE 11

| | | Concentration |
|---|---|---|
| Phase | Ingredients | (%) |
| A1 | Water | 20 |
| A1 | Terephthylidene Dicamphor Sulfonic Acid | 0.9 |
| | (Mexoryl SX from Noveal) | |
| A1 | Triethanolamine | 0.35 |
| A1 | Nicotinamide (ThermoFisher) | 2.5 |
| A1 | Sodium Salicylate (Sigma Aldrich) | 0.5 |
| A2 | Xanthan gum | 0.1 |
| A2 | Glycerol | 6 |
| A2 | Dipropylene Glycol (CABB Group GmbH) | 5 |
| A2 | Acrylates/C10-30 Alkyl Acrylate Crosspolymer | 0.25 |
| | (Pemulen TR-1 polymer from Lubrizol) | |
| A2 | Water | 22 |
| B1 | Stearic acid | 1.5 |
| B1 | Glyceryl Stearate (and) PEG-100 Stearate | 1.3 |
| | (Arlacel 165-FP-PA-(SG) from Croda) | |
| B1 | Homosalate | 6 |
| B1 | Butyl Methoxydibenzoylmethane (Parsol 1789 | 4 |
| | from DSM Nutritional Products) | |
| B1 | Ethylhexyl triazone (Uvinul T150 from BASF) | 1.8 |
| B1 | Ethylhexyl Salicylate (Parsol EHS from DSM | 5 |
| | Nutritional Products) | |
| B1 | Isopropyl Palmitate (BASF) | 7 |
| B1 | Diisopropyl Sebacate (Dub Dis from Stearinerie | 1 |
| | Dubois) | |
| B1 | Caprylyl glycol | 0.4 |
| B2 | Potassium Cetyl Phosphate (Amphisol K from | 1 |
| | DSM Nutritional Products) | |
| B2 | pH adjuster | qs pH 6.3 |
| C1 | Propylene Carbonate (Merck) | 6 |
| C1 | Methoxypropylamino cyclohexenylidene | 3 |
| | ethoxyethylcyanoacetate (Compound 25) | |
| C2 | Ethanol (Sigma Aldrich) | 4 |

Phases A1, B1 and C are prepared separately by mixing the ingredients which make up each phase, heating at 65° C. under magnetic stirring, until a clear mixture is obtained.

The ingredients of phase B2 are introduced under stirring into phase B1 until a fine dispersion of potassium cetyl phosphate is obtained.

Phase A2 is prepared by dispersing the gelling agents in glycols, before adding water and mixing with an Ultraturrax mixer at 27 000 rpm for 5 min.

Phase C is prepared by introducing compound 25 into the propylene carbonate under mechanical stirring and while heating at 65° C. until a clear mixture is obtained. The ethanol is then incorporated once the mixture has returned to ambient temperature.

Phases A1 and A2 are introduced into the phase B1+B2 by mixing with the Ultraturrax mixer at 27 000 rpm for 5 min to obtain an emulsion. Phase C is then incorporated by mixing with the Ultraturrax mixer at 27 000 rpm for 5 minutes.

1. A cosmetic or dermatological composition comprising:
a) at least one merocyanine corresponding to formula (3) below, and also the geometrical isomer forms thereof: 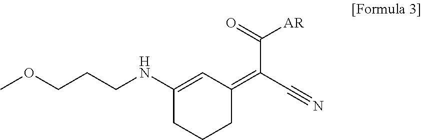
wherein:
A is —O— or —NH—;
R is a C₁-C₂₂ alkyl group, a C₂-C₂₂ alkenyl group, a C₂-C₂₂ alkynyl group, a C₃-C₂₂ cycloalkyl group or a C₃-C₂₂ cycloalkenyl group, it being possible for said groups to be interrupted by one or more O; and
b) dipropylene glycol.

2. The cosmetic or dermatological composition as claimed in claim 1, wherein the merocyanines of formula (3) are chosen from the following compounds and also the geometrical isomer forms, in particular E/E or E/Z geometrical isomer forms, thereof:
TABLE 1

| | |
|---|---|
| 14 | 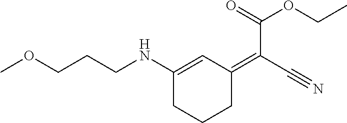 [SMILES: CCOC(=O)/C(C#N)=C1\C=C(NCCCOC)CCC1] |
| | ethyl (2Z)-cyano{3-[(3- |
| | methoxypropyl)amino]cyclohex-2-en-1- |
| | ylidene}ethanoate. |
| 15 | 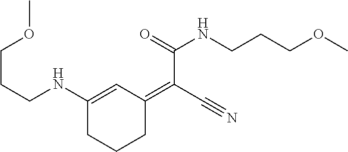 [SMILES: COCCCNC(=O)/C(C#N)=C1\C=C(NCCCOC)CCC1] |
| | (2Z)-2-cyano-N-(3-methoxypropyl)-2-{3- |
| | [(3-methoxypropyl)amino]cyclohex-2-en-1- |
| | ylidene}ethanamide |
| 25 | 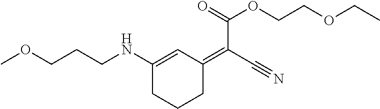 [SMILES: CCOCCOC(=O)/C(C#N)=C1\C=C(NCCCOC)CCC1] |
| | 2-ethoxyethyl (2Z)-cyano{3-[(3- |
| | methoxypropyl)amino]cyclohex-2-en-1- |
| | ylidene}ethanoate |
| 27 | 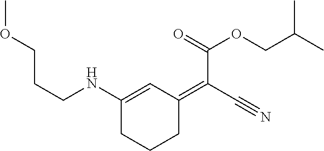 [SMILES: COCCCNC1=C/C(=C(/C#N)C(=O)OCC(C)C)CCC1] |
| | 2-methylpropyl (2Z)-cyano{3-[(3- |
| | methoxypropyl)amino]cyclohex-2-en-1- |
| | ylidene}ethanoate |
| 29 | 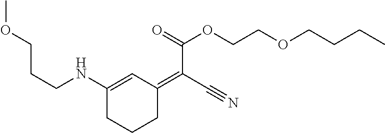 [SMILES: CCCCOCCOC(=O)/C(C#N)=C1\C=C(NCCCOC)CCC1] |
| | 2-butoxyethyl (2Z)-cyano{3-[(3- |
| | methoxypropyl)amino]cyclohex-2-en-1- |
| | ylidene}ethanoate |
| 31 | 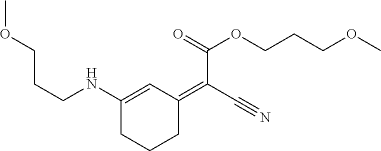 [SMILES: COCCCNC1=C/C(=C(/C#N)C(=O)OCCCOC)CCC1] |
| | 3-methoxypropyl (2Z)-cyano{3-[(3- |
| | methoxypropyl)amino]cyclohex-2-en-1- |
| | ylidene}ethanoate. |
| 37 | 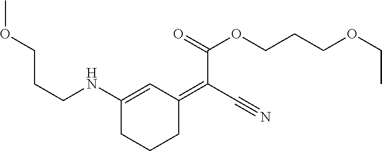 [SMILES: CCOCCCOC(=O)/C(C#N)=C1\C=C(NCCCOC)CCC1] |
| | 3-ethoxypropyl (2Z)-cyano-{3-[(3- |
| | methoxypropyl)amino]cyclohex-2-en-1- |
| | ylidene}ethanoate. |

3. The cosmetic or dermatological composition as claimed in claim 1, wherein the merocyanine of formula (3) is 2-ethoxyethyl (2Z)-cyano{3-[(3-methoxypropyl)amino]cyclohex-2-en-1-ylidene}ethanoate (25) in its E/Z geometrical configuration having the following structure: 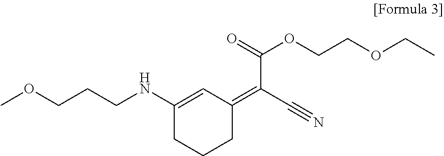 [SMILES: CCOCCOC(=O)/C(C#N)=C1\C=C(NCCCOC)CCC1]
and/or in its E/E geometrical configuration with the following structure:

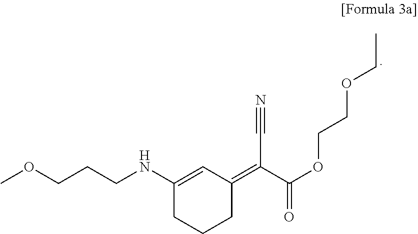 [SMILES: CCOCCOC(=O)/C(C#N)=C1/C=C(NCCCOC)CCC1]

4. The composition as claimed in claim 1, wherein the merocyanines of formula (3) and/or the geometric isomer forms thereof are present in a concentration ranging from 0.1% to 15% by weight relative to the total weight of the composition.

5. The cosmetic or dermatological composition as claimed in claim 1, wherein the dipropylene glycol is present in an amount of between 0.1% and 99% by weight of the total weight of the composition.

6. The cosmetic or dermatological composition as claimed in claim 1, comprising at least one polymer comprising monomer units of formulae (A) and (B): 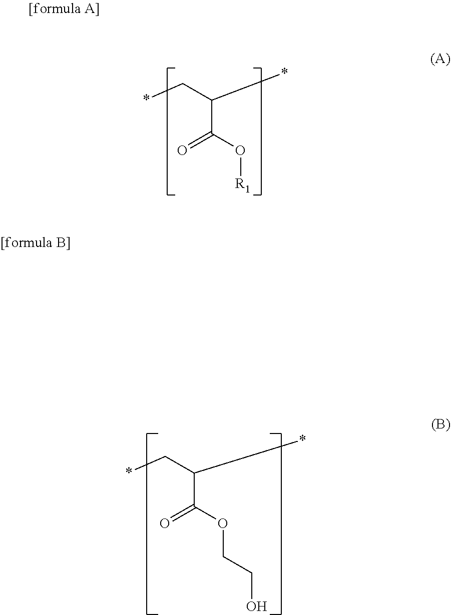
wherein:
R1, independently at each instance, is chosen from alkyl or alkenyl radicals, and
at least 60% by weight of the R₁ groups are radicals chosen from stearyl and behenyl radicals, the percentage by weight relating to the sum of all the R₁ groups present in the polymer, and
the weight ratio of the sum of all the hydroxyethyl acrylate units to the sum of all the acrylate units bearing the R₁ group ranges from 1:30 to 1:1;
and the sum of the total of units A and B is at least 95% by weight relative to the total weight of the polymer,
the polymer having a number-average molecular weight Mn ranging from 2000 to 9000 g/mol.

7. The cosmetic or dermatological composition as claimed in claim 6, wherein, in the additional polymer, R₁ is constituted of an alkyl radical.

8. The composition as claimed in claim 6, wherein, in the additional polymer, at least 70% by weight of the R₁ groups are behenyl or stearyl radicals.

9. The cosmetic or dermatological composition as claimed in claim 6, wherein, in the additional polymer, all the R₁ groups are stearyl or behenyl radicals.

10. The cosmetic or dermatological composition as claimed in claim 6, wherein, in the additional polymer, said weight ratio ranges from 1:15 to 1:1.

11. The cosmetic or dermatological composition as claimed in claim 6, wherein the additional polymer has a number-average molecular weight Mn ranging from 5000 to 9000 g/mol.

12. The cosmetic or dermatological composition as claimed in claim 6, wherein the additional polymer has a melting point ranging from 40° C. to 70° C.

13. The cosmetic or dermatological composition as claimed in claim 6, wherein, in the additional polymer, at least 60% by weight of the R₁ groups are stearyl radicals, and said additional polymer has a melting point ranging from 40 to 60° C.

14. The cosmetic or dermatological composition as claimed in claim 6, wherein, in the additional polymer, at least 60% by weight of the R₁ groups are behenyl radicals, and said additional polymer has a melting point ranging from 60° C. to 70° C.

15. The cosmetic or dermatological composition as claimed in claim 1, comprising at least one alkyl or alkylene carbonate.

16. The cosmetic or dermatological composition of claim 14, wherein the alkylene carbonate(s) are chosen from those of formula below: 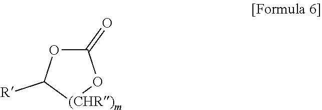
wherein:
R′ denotes a hydrogen atom, a linear or branched C₁-C₆ alkyl radical or a linear or branched C₁-C₄ hydroxyalkyl radical, preferably R′ denotes a hydrogen atom, a linear or branched C₁-C₄ alkyl radical or a linear or branched C₁-C₂ hydroxyalkyl radical;
R″ represents a hydrogen atom; a linear or branched C₁-C₆ alkyl radical or a linear or branched C₁-C₄ hydroxyalkyl radical;
m is equal to 1, 2 or 3.

17. The cosmetic or dermatological composition as claimed in claim 16, wherein the R′ radical represents a hydrogen atom, a linear or branched C1-C4 alkyl radical or a linear or branched C1-C2 hydroxyalkyl radical.

18. The cosmetic or dermatological composition as claimed in claim 16, wherein R″ represents a hydrogen atom, a linear or branched C1-C2 alkyl radical or a linear or branched C1-C2 hydroxyalkyl radical.

19. The cosmetic or dermatological composition as claimed in claim 15, wherein the alkylene carbonate used is propylene carbonate.

20. The cosmetic or dermatological composition as claimed in claim 1, also comprising one or more additional UV-screening agents.

21. A non-therapeutic cosmetic process for caring for and/or making up a keratin material, comprising the application, to the surface of said keratin material, of at least one cosmetic or dermatological composition as defined in claim 1.

22. A method for solubilizing a merocyanine corresponding to the following formula (3) and also their geometrical isomer forms: 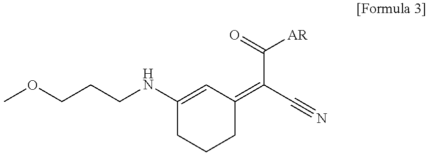
in which:
A is —O— or —NH—;
R is a C₁-C₂₂ alkyl group, a C₂-C₂₂ alkenyl group, a C₂-C₂₂ alkynyl group, a C₃-C₂₂ cycloalkyl group or a C₃-C₂₂ cycloalkenyl group, it being possible for said groups to be interrupted by one or more O in the fatty phase and/or in the aqueous phase of a composition, which comprises including dipropylene glycol in the composition.
